(19) United States
(12) Patent Application Publication
(10) Pub. No.: US 2003/0148286 A1
(43) Pub. Date: Aug. 7, 2003

(54) Method for normalizing the relative intensities of detection signals in hybridization arrays

(76) Inventors: Anne-Marie Larose, Montreal (CA); Benoit LeBlanc, Montreal (CA); Rino Camato, St-Leonard (CA)

Correspondence Address: MERCHANT & GOULD PC, P.O. BOX 2903, MINNEAPOLIS, MN, 55402-0903, US

(21) Appl. No.: 10/030,846

(22) PCT Filed: Dec. 21, 2001
(86) PCT No.: PCT/CA01/01860

(30) Foreign Application Priority Data
Dec. 27, 2000 (CA) 2,327,527

(51) Int. Cl. C12Q 1/68; G06F 19/00; G01N 33/48; G01N 33/50

(52) U.S. Cl. 435/6; 702/20

(57) ABSTRACT

The present invention relates to rRNA-derived cDNA used as an internal standard or control to achieve normalization of hybridization signal detection in microarray biochip technology. Analysis of data obtained from a laser scanner during DNA microarray experiments first requires image processing. However, the data generated for the arrayed genes must be normalized before differentially expressed genes can be identified. Normalization is necessary to compensate for differences in labelling and detection efficiencies for the labels and for differences in the quantity of starting RNA from the samples examined in the assay. Because of its relatively invariant expression across tissues and treatments, 18S and 28S ribosomal RNAs are ideal internal controls for quantitative RNA analysis. A way to circumvent the technical difficulties of using ribosomal RNA as a control, because of its overabundance relative to that of other RNAs, is described and claimed in the present application. Improved methods, arrays, and kits comprising arrays and free unlabelled ribosomal probes, are objects of this invention. The unlabelled ribosomal probes are used to compete out the excess or ribosomal nucleics present in a sample wherein all cDNA species of the sample are labelled before being placed in contact with the arrays.

### FIELD OF THE INVENTION

The present invention relates to the field of hybridization arrays. More specifically, the present invention concerns a method for normalizing signals to be compared in hybridization arrays. This novel method relies on the use of ribosomal RNA (rRNA) as an internal standard and allows approximation of the relative abundance of multiple mRNAs as well as direct comparisons between any two specific RNA samples.

### BACKGROUND OF THE INVENTION

In DNA microarray experiments, one of the more popular ways to control for spotted DNA quantity and surface chemistry anomalies involves the use of two-color fluorescence (see, refs. 4, 5). For example, a Cy3 (green)-labelled probe prepared from healthy tissue could be used as a control to examine expression profiles of a Cy5 (red)-labelled probe prepared from a tumor tissue. The normalized expression values for every gene would then be calculated as the ratio of experimental expression to control expression. This method can obviously eliminate much (but not all) experimental variation by allowing two samples to be compared on the same chip because there is enough DNA on each spot that both test and reference cDNAs can hybridize to it at once without interference. More sophisticated three-color experiments are also possible in which one channel serves as a control for the amount of spotted DNA, and channels two and three allow two samples to be compared to this control and to each other (see ref. 5).

In addition to the local normalization method described above, more general methods are also available in the form of control spots on the slide. With a set of control spots, it is possible to control variations in overall slide quality or scanning differences.

Applicable normalization strategies are based on some underlying assumptions regarding the data and the strategies used for each experiment. These strategies must therefore be adjusted to reflect both the system under study and the experimental design. A primary assumption is that for either an entire collection of arrayed genes or some subset of the genes (such as housekeeping genes), or for some added set of controls, the ratio of measured expression averaged over the set should be close to unity.

The need for good methods of normalisation for microarray data can not be overstated (see refs. 6, 7). Depending on the experimental design, there are three useful approaches for calculating normalization factors. The first simply relies on the total fluorescent intensity measured. The assumption underlying this approach is that the total mass of RNA labelled with either Cy3 or Cy5 is equal. While the intensity for any one spot may be higher in one channel than the other, when averaged over thousands of spots in a given array, these fluctuations average out. Consequently, the total integrated intensity across all the spots in the array should be equal for both channels. Alternatively, one could add a number of controls in increasing but equimolar concentrations to both labeling reactions, and the sum of the intensities for these spots should be equal.

A second approach uses linear regression analysis. For closely related samples, one would expect many of the genes to be expressed at nearly constant levels. Consequently, a scatter plot of the measured Cy5 versus Cy3 intensities should have a slope of one. Measured intensities for added equimolar controls should behave similarly. Under this assumption, one can use regression analysis techniques to calculate the slope which is used to rescale the data and adjust the slope to one.

A third approach has been described by Chen et al (1997) (ref. 1). In it, it is assumed that a subset of housekeeping genes exists and that for these genes the distribution of transcription levels should have some mean value and standard deviation that are independent of any particular sample. In this case, the ratio of measured Cy5 to Cy3 ratios for these genes can be modeled and the mean of the ratio adjusted to 1. Chen and his collaborators describe an iterative procedure to achieve this normalization. Quackenbush and collaborators (ref. 2) have implemented their own algorithm and a variation thereof that uses the entire data set in a data visualization and analysis tool called TIGR ArrayViewer. Other statistical methods of determining data accuracy have been described (ref. 3, 11).

The above procedures describe array-based measures that can be used to normalize data. However, even with multiple colour fluorescence and control spots, undesired experimental variation can contaminate expression data. It is also possible that some or all of the physical normalization techniques are missing from the experiment, in which case it is even more important to find additional means of normalization.

The use of internal standards overcomes these problems. Using an exogenously added standard has the advantage of giving the user absolute control over the amount of template added, with no variation between samples. Using an exogenous standard does not, however, control differences in the quality of the starting RNA in a reverse transcription reaction. If there are differences in the levels of integrity of the RNA between otherwise identical samples, the yield of specific reverse transcriptase products will reflect this variation, although the external standards will still appear identical. For this reason, as well as for simplicity and reproducibility, an endogenous RNA standard should be favoured in microarray experiments.

Theoretically, an ideal endogenous standard for a DNA microarray would be a transcript whose expression does not vary during the cell cycle, between cell types, or in response to the experimental treatments that one wishes to examine. Additionally, for an endogenous standard to be valid in a microarray it is crucial that it be of a similar relative abundance as the test and reference (or target) transcripts in the microarray. Unfortunately, such a molecule does not exist and there are serious limitations to the standards currently in use. For example, although beta-actin is a frequently used standard (refs 9, 10), its level of expression varies significantly from tissue to tissue.

For DNA microarray experiments, mRNA is copied into cDNA with the use of reverse transcriptase so that the relative abundance of individual mRNAs is reflected in the cDNA product. Input RNA in reverse transcription reactions is usually quantified by spectrophotometry. The RNA that is used in a typical pre-reverse transcription reaction is total RNA, 80% of which is ribosomal RNA. The mRNA component of total cellular RNA can vary from 2% to 5% depending on the tissue, the remainder of the RNA consisting of tRNA or small nuclear RNAs. Therefore, even if a transcript is invariant (as expressed as a percentage of mRNA), its relative abundance would still vary when considered as a percent of the total input RNA from different source tissues. Since the majority of the RNA is rRNA, the level of rRNA remains essentially constant from sample to sample. Because 18S and 28S rRNA make up the majority of optically absorbent material at OD₂₆₀ₙₘ, they should make ideal invariant controls. In fact, 18S and 28S transcripts are frequently used as internal controls in northern hybridization, RNAse protection and quantitative RT-PCR assays (see ref. 8). However, the overwhelming abundance of rRNA is a major limitation to its utility as a control in DNA microarray experiments.

In U.S. Pat. No. 6,057,134, Ambion describes a method to perform RT-PCR™ which allows an invariant transcript of any relative abundance such as an 18S, 28S, or 5S ribosomal RNA, actin, or glyceraldehyde 3-P phosphate dehydrogenase RNA to be used as a control for any other transcript. This allows two targets of vastly different abundance to be quantified simultaneously in a multiplex RT-PCR™ reaction. Ambion uses blocked primers, or Competimers™, that compete with the unmodified primers for binding to a DNA template but cannot be used as primers for extension by a DNA polymerase Thus, at each extension step in PCR™, a percentage of template is unavailable for amplification. By increasing the ratio of Competimers™ to primers in a PCR™ reaction, the amplification efficiency of an amplicon can be reduced so that the linear phase of accumulation of PCR™ product matches that of a less abundant target in multiplex PCR™.

For a control to be usable for microarray hybridization, the intensity of the signal should be in the same dynamic range as the cDNA under evaluation. rRNA-derived cDNA has never previously proved useful as a control for microarrays probably because it is thousands of times too abundant compared to specific cDNA.

### OBJECTS OF THE INVENTION

An object of the present invention is therefore to provide an improved method for providing an internal standard for normalizing the relative intensities of signals in hybridization arrays, an improved method for normalizing per se and a method of hybridizing making use of the improved normalization.

### SUMMARY OF THE INVENTION

More specifically, in accordance with the present invention, there is provided an improved method for providing an internal standard for normalizing the relative intensities of signals in hybridization arrays that is based on the use of ribosomal RNA (rRNA) as this internal standard. Ribosomal RNA has been found to be particularly suitable for this purpose because its abundance, in terms of percentage of total RNA, does not vary through the cell cycle or with a particular treatment.

The method of the present invention may be summarized as follows. On a given DNA microarray, for example, an oligonucleotide specifically recognizing a sequence contained in ribosomal RNA is spotted along with the other DNA probes used to analyze gene expression, as is usual with this technique. The spots therefore essentially consist of capture probes. Ribosomal RNA, being of relatively invariant quantity in terms of percentage relative to total RNA provides a stable quantitative control to evaluate the quantity of other types of RNA. However, since it is also found in massive amounts relative to other RNAs, its level of detection by the technique must be toned down while remaining accurate. To that end, an experimentally-defined quantity of oligonucleotides carrying the same sequence as that of the oligonucleotide capture probe found on a spot of the microarray is added to the hybridization mixture so that the excess signal coming from the labelled rRNA (or from the cDNA generated from the rRNA, if cDNA hybridization is the method selected) is competed out and the signal detected for it is reduced to a range compatible with that of the signal for the other labelled RNAs.

Specifically, the present invention provides a novel method for providing an internal standard for normalizing the relative intensities of signals on a hybridization array, comprising:

adding a known quantity of an unlabelled ribosomal nucleic acid competitor probe into a hybridization buffer suitable for the array experiment, the competitor probe characterized in that it has the same sequence as at least portion of a capture probe present in the array for immobilizing ribosomal nucleic acids thereon; and

allowing the competitor probe to compete with a ribosomal capture probe for hybridization to a suitably labelled RRNA-derived cDNA of a cDNA sample, such that a hybridization signal of labelled rRNA-derived cDNA is decreased to a suitable signal dynamic range of detection and the rRNA-derived cDNA of the sample becomes a suitable internal standard for the hybridization array.

The method of the present invention may further include:

determinating the quantity of hybridized rRNA-derived cDNA; and

comparing the quantity of hybridized rRNA-derived cDNA against standard curves to determine the quantity of cDNA in said sample.

The present invention further provides a normalization method, wherein the above steps for obtaining an internal standard are reproduced for a test sample using a first label, and for a suitably-labelled reference sample using a second label, and the quantity of hybridized rRNA-derived cDNA originating from the test sample is compared to the quantity of rRNA-derived cDNA originating from the reference sample hybridizing to the same capture probe to provide a normalization factor.

The present invention further provides a hybridization array, wherein the above steps for normalizing are reiterated and the normalization factor is used to correct a hybridization signal provided by the binding of a target cDNA of the test sample labelled with the first label to a capture probe specific to said target, which correction makes said hybridization signal directly comparable to a hybridization signal provided by the binding of the same target of the reference sample labelled with the second label to the same capture probe specific to said target.

In a preferred embodiment, the rRNA competitor probe is present in a concentration that is about 5 to about 100 times that of the capture probe.

The rRNA-derived cDNA may be labelled by any suitable means, such as by 3′ addition of phosphate, or labelling with cyanines, biotin, digoxygenin, fluorescein, a dideoxynucleotide, an amine, a thiol, an azo (N₃) group or fluorine, or any other form of label.

An array comprising a plurality of spotted cDNA capture probes for binding ribosomal nucleics, alone or in combination with the competitor ribosomal probe in a separate component are further objects of this invention. The method of the present invention is suitable for use in high-throughput screening experiments.

It may be used for any type of array experiment, including but not limited to the identification of sequences found in the open reading frame of genes coding for transcription factors, such as c-Rel, E2F-1, Egr-1, ER, NFκB, p50, p53, Sp1 and YY1.

Other objects, advantages and features of the present invention will become more apparent upon reading of the following non restrictive description of preferred embodiments thereof, given by way of example only with reference to the accompanying drawings.

### BRIEF DESCRIPTION OF THE DRAWINGS

In the appended drawings:

FIG. 1: A summary view of the described technology. Any given pool of total cellular RNA is usually composed of 80% ribosomal RNA (rRNA) and 20% messenger RNA (mRNA) and small nuclear RNAs. mRNA (except for the histone genes) is polyadenylated while rRNA never is. Making cDNA from both types of RNA by reverse transcription is possible if using a poly dT primer for mRNA (producing mRNA-derived cDNA, shown by solid arrows) and a specific primer for rRNA (producing rRNA-derived cDNA, shown by dashed arrows). Analysis of mRNA by microarray using the constant rRNA as a standard is made difficult by the relative overabundance of rRNA relative to mRNA; this problem is circumvented by adding to the hybridization mix a rRNA competitor probe which has the same sequence as the microarray's rRNA-cDNA capture probe (both shown as lines marked with an “r”). By sequestering the excess rRNA-derived cDNA, the competitor probe brings down the level of hybridizable and hybridized rRNA-derived cDNA to usable levels.

FIG. 2: Human ribosomal DNA complete repeating unit (GB accession number #U13360). ETS: externally transcribed spacer. ITS: internally transcribed spacer. IGS: intergenic spacer. The position of a few rRNA probes is shown.

FIG. 3: Illustration of spotted DNA capture probes on the slide. The slide used for the described experiment carries 12 probe blocks, identified 1 to 12. In each block there are 7 rows and 16 columns of spots. Each DNA capture probe was spotted in duplicate in an adjacent column (i.e., all odd columns correspond to a duplicate column) so there are 8 different DNA probes in a column. There are a total of 1344 spots on the slide, corresponding to duplicates of 463 different DNA capture probes and 209 negative controls (no DNA probe).

FIG. 4: Cohybridization of labelled cDNA from Jurkat (reference sample: Cy3-green) and Jurkat-TPA (test sample: Cy5-red). Ratio images exported from GenePix Pro 3.0 (Axon Instruments Inc.) as JPEG (or TIFF) files are 24-bit RGB color.

FIG. 5: Cohybridization of labelled cDNA from Jurkat (Cy3-green) and Jurkat-TPA (Cy5-red). Five (5) ng of rRNA competitor probe 2 was added to the hybridization mix to compete for the hybridization of the rRNA-derived cDNA to the attached rRNA cDNA capture probe 2. Ratio images exported from GenePix Pro 3.0 (Axon Instruments Inc.) as JPEG (or TIFF) files are 24-bit RGB color.

FIG. 6: Cohybridization of labelled cDNA from Jurkat (Cy3-green) and Jurkat-TPA (Cy5-red). Fifty (50) ng of rRNA competitor probe 2 was added to the hybridization mix to compete for the hybridization of the rRNA-derived cDNA to the attached rRNA cDNA capture probe 2 (which has the same sequence as rRNA competitor probe 2). Ratio images exported from GenePix Pro 3.0 (Axon Instruments Inc.) as JPEG (or TIFF) files are 24-bit RGB color.

### DETAILED DESCRIPTION OF THE PREFERRED EMBODIMENT

Glossary

In order to provide a clear and consistent understanding of terms used in the present description, a number of definitions are herein provided.

Array: In the context of this invention, an array is a set of different spotted DNA consisting of capture probes for target nucleic acids. Such an array is described in U.S. Pat. No. 5,700,637.

Complementary DNA (cDNA): DNA that has been synthesized from RNA by the effect of the enzyme reverse transcriptase, converting RNA bases into their complements (A to T, U to A, G to C, C to G).

Cy3, Cy5: Non-radioactive fluorescent dyes from Amersham Pharmacia Biotech that are widely used for labeling DNA in microarray experiments.

Feature: A feature is a spot (typically of DNA) on a slide. The collection of such features is called a microarray.

Hydridization : The process of joining two complementary strands of DNA, or one strand each of DNA and RNA, to form a double-stranded molecule.

Messenger RNA (mRNA): RNA that is used to direct the protein synthesis that is part of gene expression. It represents but a small fraction of the total RNA found in a cell.

mRNA-derived cDNA: cDNA synthesized from a mRNA template using reverse transcriptase and a mRNA-specific primer.

Microarray-sequestered DNA or DNA capture probe: DNA (single-stranded or double-stranded) that are anchored onto the solid surface of a microarray. (See fuller description of microarrays immediately following this Glossary.)

Oligonucleotide: A short strand of single-stranded DNA, typically composed of up to 50 bases.

Pixel Intensity: The raw intensity of a pixel on a GenePix (Axon Instrument Inc.) single-wavelength or ratio image, falling in a range from 0 to 65535.

PMT: Photomultiplier tubes in scanners used to analyze array images. These array images are the end products of comparative hybridization experiments.

Ratio Image : The ratio image is an RGB (Red-Green-Blue) overlay image. In this image, wavelength #1 (635 nm) is mapped to the green channel of the RGB image, and wavelength #2 (532 nm) is mapped to the red channel. Superimposing these two images onto each other results in a third, composite image, whose color is a blend of the red and green signals.

Ratio of medians: The ratio of medians is the ratio of the background subtracted median pixel intensity at the second wavelength to the background subtracted median pixel intensity at the first wavelength.

Reference cDNA: this cDNA originates from a reference sample that is used for comparison with another one, called test cDNA obtained from a test sample. The reference cDNA serves as a control against which test cDNAs may be compared to quantify changes in the level of expression of any mRNA found in the test sample. Typically, the reference cDNA is labelled with Cy3-dCTP (green fluorescent label) when a fluorescent label is used.

RGB: Red-Green-Blue color.

Ribosomal RNA (rRNA): structural RNA found in the ribosomes. It is the most abundant form of RNA in the cell and does not vary significantly.

rRNA-cDNA probe: a probe which is designed to hybridize to the rRNA-derived cDNA found in the hybridization mixture. This probe may be the capture probe, which may have the same sequence as the rRNA competitor probe (see below) so as to compete with it for the target rRNA-derived cDNA.

rRNA competitor probe: a DNA oligonucleotide with the same sequence as part of a ribosomal RNA-cDNA sequence and capable of competing with the microarray capture probe for hybridization with a rRNA-derived cDNA. This oligonucleotide has the role of competing for the limited space available on the rRNA cDNA capture probe bound to the microarray, thus reducing the quantity of rRNA-derived cDNA which can be retained on the microarray and thus allowing the use of rRNA-derived cDNA as an <<internal standard>>.

rRNA-derived cDNA: cDNA synthesized from a rRNA template using reverse transcriptase and a rRNA-specific primer.

Saturation : Saturation refers to the overloading of the photodetection circuitry. Saturation can be reduced by reducing the amount of light that is reaching the PMTs, which is done by reducing the amount of incident laser light. In practice, this is accomplished by reducing the voltage of the PMT, which reduces its gain. Saturating pixels in GenePix 1.0 are shown as white pixels in the raw wavelength images.

Spotted DNA: Known DNA capture probe that is spotted onto a microarray slide and used to identify the nucleic acids present in unknown samples (test and reference). The spotted DNA could be oligonucleotide or cDNA.

Test cDNA: cDNA from a cell sample that is to be tested, in comparison with a reference sample. Typically, the test cDNA is labelled with Cy5-dCTP (red fluorescent label) when a fluorescent label is used.

Microarrays are made from a collection of purified DNAs. A drop of each type of DNA in solution is placed onto a specially-prepared glass microscope slide by an arraying machine. The arraying machine can quickly produce a regular grid of thousands of spots in a square about 2 cm on a side, small enough to fit under a standard slide cover slip. The DNA in the spots is bound to the glass to keep it from washing off during the hybridization reaction. The choice of DNA to be used within the spots on a microarray's surface determines which genes can be detected in a comparative hybridization assay. These DNA probes could be synthetic oligonucleotides or PCR amplified DNA (hence the terms “oligo microarray” and “cDNA microarray”).

The invention relates to rRNA used as an internal standard for the normalization of the fluorescence intensities in microarray analysis experiments. This can provide an estimate of relative abundance of multiple mRNAs and allow direct comparison between two RNA samples.

Use of rRNA for normalization provides a sound method of identifying differentially expressed genes between two samples because its percentage of abundance in total RNA does not vary through the cell cycle or with a particular treatment.

In order to detect the difference in gene expression between two samples on a single microarray slide, the RNA should be reverse transcribed to cDNA and labelled with two different fluorophores prior to cohybridizing both samples to the same slide and same spots simultaneously. There are several techniques that allow labeling of cDNA. Direct labeling is done by the incorporation of a fluorescent nucleotide such as, for example, Cy3-dCTP (green) or Cy5-dCTP (red) (from Amersham-Pharmacia Biotech), during the reverse transcription reaction. Other protocols may be used for labeling the cDNA following the reverse transcription reaction (indirect labeling). Alternatively, the cDNA can be used for RNA amplification involving T7 polymerase. This method relies on attaching a T7 promoter sequence to the reverse transcriptase primer used for synthesis of the first cDNA strand. After second strand cDNA synthesis, one can generate amplified RNA (aRNA) using T7 RNA polymerase and the double-stranded cDNA molecules as targets for the linear amplification. Those targets can then be labelled directly or indirectly.

In the present invention, the reverse transcriptase reaction for the cDNA labeling step involves the use of two kinds of reverse transcriptase primers in the same reaction: an oligo-dT and specific primers for rRNA (5.8S, 1 8S or 28S rRNA). One set of RNA to be reverse transcribed is all the polyA+ mRNA that is present in the RNA sample, the other set is the rRNA. Both sets are labelled in the same sample with the same label. Random short primer like random hexamers or sets of specific primers could also be used as alternative methods to reverse transcribe all the polyA+ mRNA.

In a typical experiment, the reference cDNA is labelled with Cy3 and the test cDNA is prepared in the presence of Cy5. Both of these cDNA populations are hybridized to the same spotted DNA capture probes on the microscope slide. After the hybridization and washing steps, the slide is scanned at the appropriate wavelengths and an image is generated for each wavelength. In the derived ratio image, a red spot indicates that the test cDNA for this feature is more abundant than the reference cDNA which means that the test cDNA is being expressed at a level higher than the reference cDNA; a yellow spot means that there is no change in the expression level between the two populations of test and reference cDNA. In order to measure changes in gene expression numerically, image analysis software like GenePix 1.0 (Axon Instruments, Inc.) extracts the intensity of a given feature (spot) from an image and performs a number of computations on the raw data. In this kind of comparative analysis, normalization is essential to compensate for variations in RNA isolation techniques, initial quantification errors, tube to tube variation in reverse transcriptase reactions and other experimental variations. That is where the present invention intervenes: normalization is possible upon correcting the green intensity and the red intensity of the spot having the internal standard capture probe to achieve a ratio of 1. This normalization therefore leads to the obtention of a correction factor that is applied to the intensities of signals specific to each reference and test samples.

The end product of a comparative hybridization experiment is a scanned array image. Saturated pixels appear when there are more photons detected than can be processed by the photomultiplier tubes (PMT) of the scanner. This occurs when the amount of hybridized target per shot is too high. Saturated pixels cannot be used for proper measurement of the signal intensity. PMT should then be set to avoid the detection of saturated pixels. As a consequence, this reduces the signal intensity of all other spots and low levels of cDNA will not be detected.

In the present invention, the hybridization step is performed with specific amounts of free rRNA-derived cDNA (competitor probe) added into the hybridization buffer so as to set up a competition for ribosomal cDNA of the test cDNA and of the reference cDNA (if the latter is part of the experiment) with the capture probe. For efficient competition, the competition probe should be nearly identical to the capture probe or have a high level of overlapping sequences therewith. The hybridization efficiency of the rRNA-derived cDNA with the capture probe can be predictably and reproducibly altered. Reducing the hybridization of these internal and abundant targets in microarray experiments has the effect of generating a signal intensity in the same dynamic range of detection as the less abundant targets in microarrays.

The competition is important because the control must be detected at a level similar to the test transcript. If one target is present at a significantly higher concentration than the other, the PMT (laser voltage) has to be reduced to avoid a saturated signal, with the consequence of reducing all the other signals. The ability to obtain quantitative information for low abundant mRNA will then be lost.

With the applicants' invention, the normalization factor is computed using the ratio of intensity obtained between the signal detected for the test cDNA and that of the reference cDNA. This ratio should be 1.0. For example, if the ratio is 0.8, a normalization factor of 1.25 would have to be calculated (1/0.8). The analyzed data is then corrected using this factor. If the normalization factor is greater than 2 (or less than 0.5) the slide is usually rescanned with other PMT voltage to ensure maximum data integrity.

Results

The applicants used the products and protocols that are described herein, which results in proper normalization.

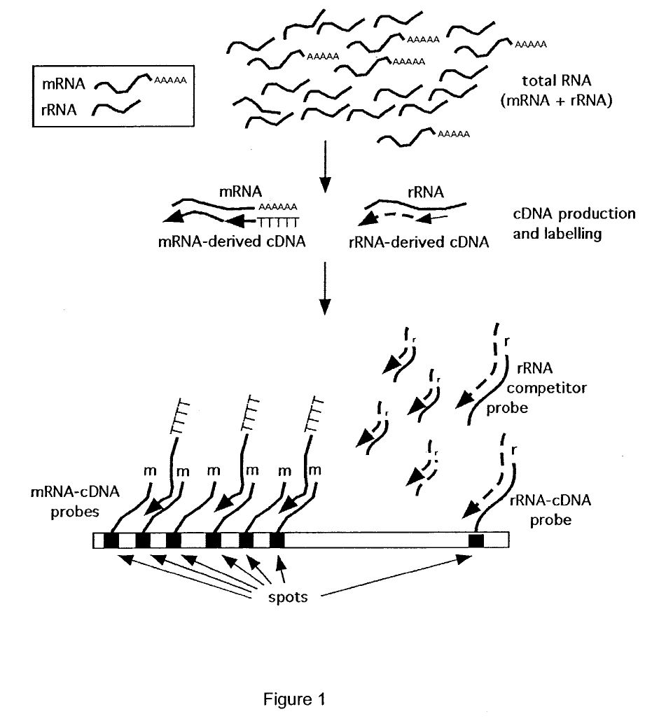

FIG. 1 illustrates how a given sample (reference or test) is labelled and hybridized to capture probes (a plurality of specific cDNA probed spots and one internal standard probe spot). The labelled ribosomal cDNA is mixed with a competitor probe that is here identical to the capture probe.

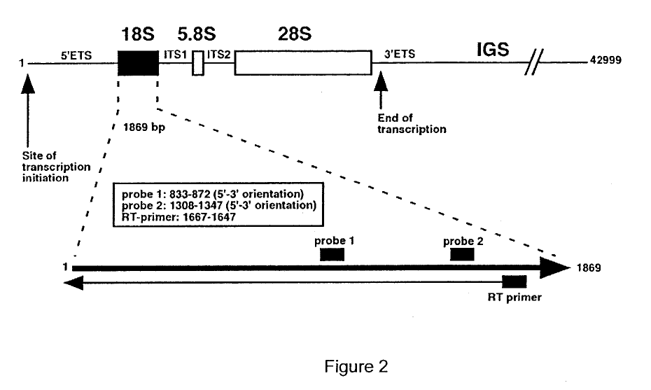

FIG. 2 illustrates the organization of the rDNA locus. The microarray was made from a collection of synthetic DNA oligonucleotides as DNA probes.

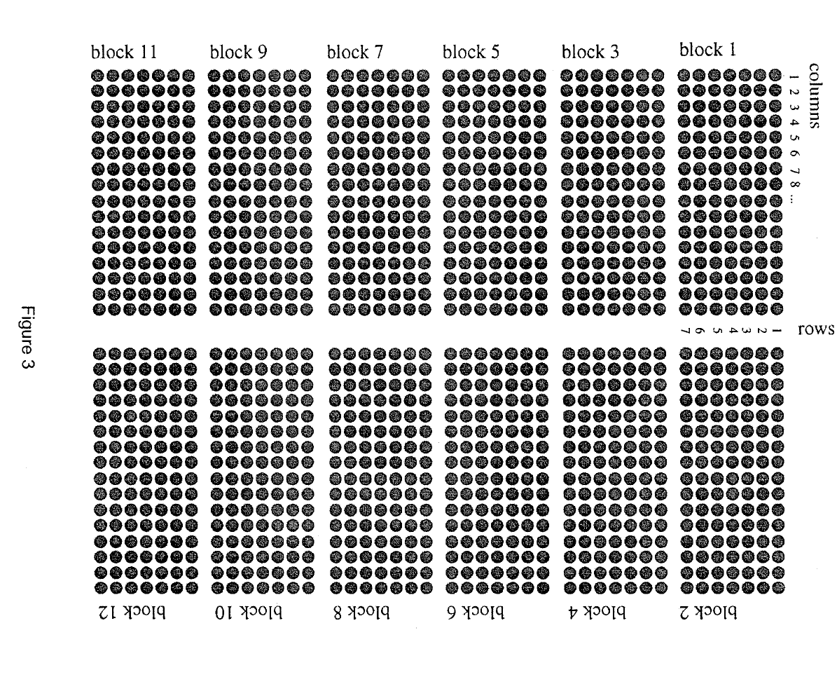

FIG. 3 illustrates the positions of spotted DNA capture probes on the slide. In order to use the cDNA made from rRNA for normalisation, a DNA capture probe having a sequence that is complementary to the rRNA-derived cDNA has also been spotted on the array slide.

Table 1 shows the sequences of two DNA-probes designed for that purpose. 3D-Link Activated slides from Surmodics Inc. were used according to the supplier's protocol for the covalent attachment of the 5′ amino modified oligonucleotides and prehybridization treatment of the slides. On the DNA microarray used here, each spot contains approximately 0.15 ng of bound DNA probe.

The cDNA for microarray analysis was prepared from RNA templates by incorporation of fluorescent-labelled deoxyribonucleotides during first strand cDNA synthesis. 10 μg of total RNA extract from Jurkat and Jurkat-TPA cell lines (Geneka Biotechnology) was used. Priming of cDNA synthesis was performed using 2 μg of oligo (dT). For each labeling reaction, 50 ng of 18S primer were included to allow reverse transcription of the 18S rRNA. Table 1 shows the sequences of the 18S reverse transcriptase primer. In this experiment, labelled reference cDNA from Jurkat total RNA was prepared using Cy3-dCTP while Jurkat-TPA total RNA was reverse transcribed and labelled using Cy5-dCTP (Amersham Pharmacia Biotech) to produce labelled test cDNA. Reverse transcriptase reactions were performed using the Superscript II reverse transcriptase (LifeTechnologies) enzyme according to the supplier's protocol.

For the hybridization and washing steps the following conditions were used (optimized conditions for 3D-Link Activated slides, Surmodics Inc.). Labelled cDNAs were cohybridized in 5×SSC-0.1% SDS buffer for 16 hours at 45° C. Washing was performed by incubating slides two times 15 minutes in 2×SSC-0.1% SDS at 45° C., one time 5 minutes in 0.2×SSC at room temperature and one time 5 minutes in 0.1×SSC at room temperature. Slides were dried by low speed centrifugation.

The test and reference cDNAs were analyzed through hybridization with the microarray-sequestered cDNA. In this type of experiment, if the test or reference cDNA contains a sequence that is complementary to the DNA on a given spot, that cDNA will hybridize to the spot, where it will be detectable by virtue of its fluorescence.

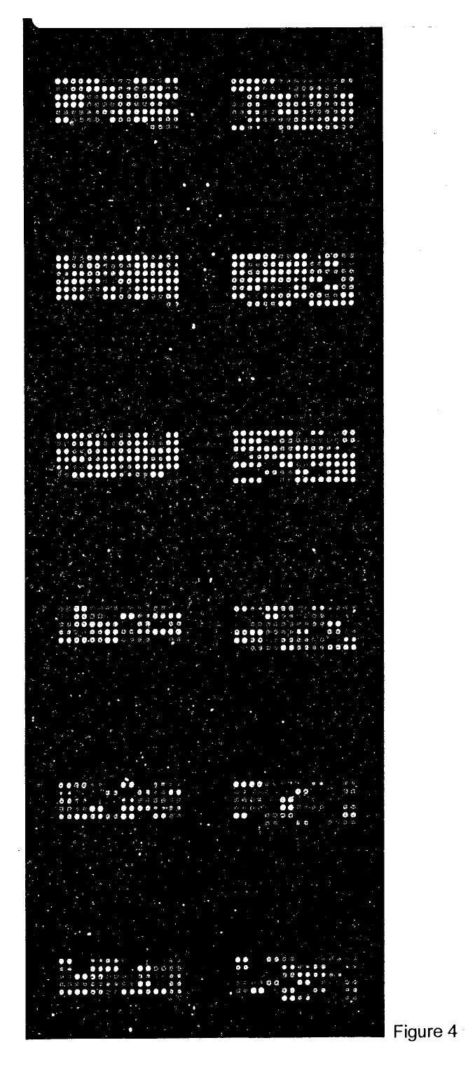

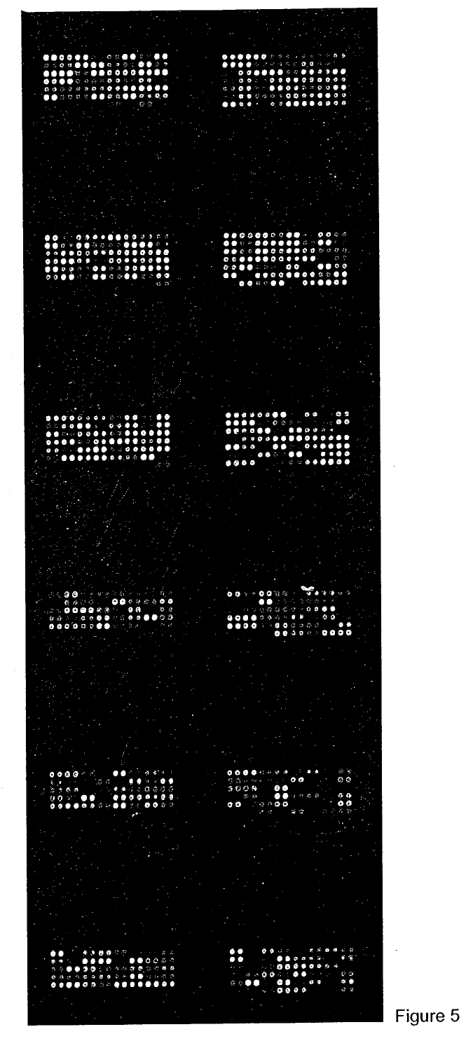

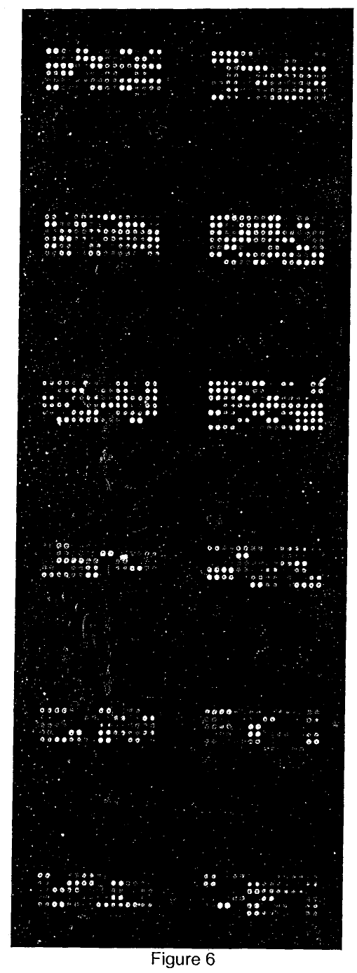

FIG. 4 shows a ratio image of a typical cohybridized cDNA with no internal standard according to the invention. The target cDNAs and the results are listed in Table 2 (see right column). FIGS. 5 and 6 show counterparts of arrays of FIG. 4 but with 5 ng and 50 ng of ribosomal competitor probe, respectively, in accordance with this invention. The results are listed in Table 2, in the middle and left columns, respectively.

Saturated spots were observed for the two rRNA cDNA probes (DNA probe 1 and probe 2). The GenePix 3.0 software (Axon Instruments Inc.) was used to extract the intensity of each feature (hybridized spot) from the image. Table 2 shows the mean value of pixel intensity for each spot. To analyse feature intensity and calculate a ratio, the local background should be subtracted from the median value of the pixel. The method used by GenePix Pro 3.0 for determining the background intensity is a local background subtraction technique. A different background is therefore computed for each individual feature-indicator and the median value of the background pixel intensities are reported (Table 2).

The end product of a comparative hybridization experiment is a scanned array image. Saturated pixels appear when there are more photons detected than the photomultiplier tubes (PMT) of the scanner can process. This occurs when the amount of hybridized cDNA to the spot is too high. Saturated pixels cannot be used for proper measurement of the signal intensity. PMT should then be set to avoid the detection of saturated pixels. As a consequence, this reduces the signal intensity of all other spots, and lower levels of cDNA will not be detected.

Because of the high abundance of the rRNA-derived cDNA relatively to the mRNA-derived cDNA, it is important to reduce its hybridization to the microarray-sequestered DNA. In this invention, the applicants compete the hybridization of the rRNA-derived cDNA to the microarray DNA capture probe by adding a defined amount of rRNA competitor probe in the hybridization buffer, said probe carrying the same sequence as the microarray-bound probe. Five (5) to 100 molar excess of competitor probe relative to the quantity of microarray DNA capture probe is enough to obtain a rRNA-derived cDNA signal intensity in the same dynamic range of detection as the other cDNAs (i.e., test and/or reference mRNA-derived cDNA), which are otherwise present in much lesser quantities in the reaction buffer. The amount of molar excess to be used is essentially a function of the amount of the total RNA used for the assay (for example: 0.2 to 20 μg).

In short, because of their relatively invariant expression across tissues and treatments, 18S and 28S RNA are ideal internal controls for quantitative RNA analysis by microarrays. The current invention describes how to use these rRNAs to that end by compensating, thanks to competition with specific oligos, for their overabundance relative to the mRNA of test and reference cell samples.

The overall exhaustive results of comparison of test and reference cDNAs, normalized in accordance with the method and principles of the present invention, are provided in appendix 1.

Although the present invention has been described hereinabove by way of preferred embodiments thereof, it can be modified, without departing from the spirit and nature of the subject invention, as defined in the appended claims.

TABLE 1

| | | Positions relative | Spotted position | | |
|---|---|---|---|---|---|
| Name | DNA sequences | to 5′ 18S sequence | Block | Column | Row |
| RT primer | CTTATGACCCGCACTTACTCG | 5′-1667-1647-3′ | — | — | — |
| DNA probe 1 | CCCGAGCCGCCTGGATACCGCAGCTAGGAATAATGGAATA | 5′-833-872-3′ | 8 | 1 | 5 |
| | | | 8 | 2 | 5 |
| | | | 1 | 11 | 6 |
| | | | 1 | 12 | 6 |
| DNA probe 2 | TCTCGATTCCGTGGGTGGTGGTGCATGGCCGTTCTTAGTT | 5′-1308-1647-3′ | 10 | 1 | 5 |
| | | | 10 | 2 | 5 |
| | | | 3 | 11 | 6 |
| | | | 3 | 12 | 6 |

TABLE 2

| | | | | | Hybridization with 50 ug of probe 2 as competitor | | | | |
|---|---|---|---|---|---|---|---|---|---|
| | | | | | Ratio of median value | | | | |
| | | | | | Not | | | | |
| | | | | | normalized | beta-actin | 18 S | F635 | F532 |
| Block | Column | Row | Gene Name | Probe name | — | 1.02 | — | median | median |
| 8 | 1 | 5 | 18S | probe 1 | 1.04 | 1.02 | undetectable | 27678 | 26672 |
| 1 | 11 | 6 | 18S | probe 1 | 1.00 | 0.98 | undetectable | 65217 | 65349 |
| 1 | 12 | 6 | 18S | probe 1 | 1.00 | 0.98 | undetectable | 65217 | 65352 |
| 8 | 2 | 5 | 18S | probe 1 | 0.85 | 0.83 | undetectable | 21986 | 26060 |
| 10 | 1 | 6 | 18S | probe 2 | 0.93 | 0.91 | undetectable | −73 | 33 |
| 10 | 2 | 6 | 18S | probe 2 | 1.27 | 1.25 | undetectable | −31 | 10 |
| 3 | 12 | 6 | 18S | probe 2 | 1.00 | 0.98 | undetectable | 83 | 254 |
| 3 | 11 | 6 | 18S | probe 2 | 1.02 | 1.00 | undetectable | 122 | 285 |
| 5 | 7 | 6 | Beta actin | actin 1 | 0.78 | 0.76 | undetectable | 1159 | 1791 |
| 5 | 8 | 6 | Beta actin | actin 1 | 0.88 | 0.87 | undetectable | 977 | 1351 |
| 10 | 3 | 1 | Beta actin | actin 1 | 0.87 | 0.85 | undetectable | 1674 | 2034 |
| 10 | 4 | 1 | Beta actin | actin 1 | 0.89 | 0.87 | undetectable | 1880 | 2213 |
| 4 | 3 | 1 | Beta actin | actin 1 | 0.63 | 0.62 | undetectable | 2010 | 3400 |
| 11 | 14 | 5 | Beta actin | actin 1 | 0.86 | 0.84 | undetectable | 1607 | 1981 |
| 11 | 13 | 5 | Beta actin | actin 1 | 0.91 | 0.89 | undetectable | 1760 | 2021 |
| 4 | 4 | 1 | Beta actin | actin 1 | 0.68 | 0.67 | undetectable | 1833 | 2880 |
| 6 | 1 | 1 | Beta actin | actin 2 | 0.96 | 0.94 | undetectable | 3619 | 3853 |
| 3 | 2 | 1 | Beta actin | actin 2 | 0.88 | 0.86 | undetectable | 278 | 603 |
| 4 | 2 | 1 | Beta actin | actin 2 | 0.81 | 0.80 | undetectable | 1667 | 2185 |
| 6 | 2 | 1 | Beta actin | actin 2 | 1.00 | 0.98 | undetectable | 3013 | 3092 |
| 1 | 8 | 6 | Beta actin | actin 2 | 0.75 | 0.73 | undetectable | 1641 | 2348 |
| 4 | 1 | 1 | Beta actin | actin 2 | 0.75 | 0.73 | undetectable | 1651 | 2355 |
| 3 | 1 | 1 | Beta actin | actin 2 | 0.93 | 0.91 | undetectable | 419 | 686 |
| 5 | 1 | 1 | Beta actin | actin 2 | 0.87 | 0.86 | undetectable | 530 | 827 |
| 5 | 2 | 1 | Beta actin | actin 2 | 0.79 | 0.77 | undetectable | 323 | 673 |
| 1 | 7 | 6 | Beta actin | actin 2 | 0.76 | 0.75 | undetectable | 2157 | 2986 |
| 3 | 8 | 6 | Beta actin | actin 3 | 1.41 | 1.38 | undetectable | 1765 | 1336 |
| 3 | 7 | 6 | Beta actin | actin 3 | 1.26 | 1.23 | undetectable | 2079 | 1744 |
| 11 | 2 | 1 | Beta actin | actin 3 | 1.51 | 1.48 | undetectable | 1697 | 1175 |
| 11 | 1 | 1 | Beta actin | actin 3 | 1.50 | 1.47 | undetectable | 1852 | 1299 |
| 12 | 2 | 1 | Beta actin | actin 3 | 1.22 | 1.19 | undetectable | 572 | 534 |
| 12 | 1 | 1 | Beta actin | actin 3 | 1.13 | 1.11 | undetectable | 545 | 651 |
| 10 | 2 | 1 | Beta actin | actin 3 | 1.11 | 1.09 | undetectable | 980 | 947 |
| 9 | 2 | 1 | Beta actin | actin 3 | 1.23 | 1.21 | undetectable | 1173 | 1020 |
| 10 | 1 | 1 | Beta actin | actin 3 | 0.92 | 0.90 | undetectable | 514 | 655 |
| 8 | 2 | 1 | Beta actin | actin 3 | 1.28 | 1.25 | undetectable | 991 | 808 |
| 8 | 1 | 1 | Beta actin | actin 3 | 1.36 | 1.34 | undetectable | 931 | 704 |
| 9 | 13 | 5 | Beta actin | actin 3 | 1.28 | 1.25 | undetectable | 1379 | 1128 |
| 9 | 1 | 1 | Beta actin | actin 3 | 1.43 | 1.40 | undetectable | 1330 | 976 |
| 9 | 14 | 5 | Beta actin | actin 3 | 1.51 | 1.48 | undetectable | 1946 | 1303 |
| 2 | 1 | 1 | Beta actin | actin 3 | 0.76 | 0.74 | undetectable | 1630 | 2269 |
| 2 | 2 | 1 | Beta actin | actin 3 | 0.76 | 0.75 | undetectable | 1800 | 2462 |
| 4 | 2 | 4 | 9G8 splicing | L22253_B | 0.69 | 0.68 | undetectable | 361 | 689 |
| 9 | 14 | 4 | A-Myb | X13294_B | 2.30 | 2.25 | undetectable | 197 | 64 |
| 4 | 8 | 4 | ASH1 | L08424_A | 1.33 | 1.31 | undetectable | 587 | 487 |
| 3 | 5 | 3 | BTEB | D31716_B | 3.88 | 3.80 | undetectable | 332 | 33 |
| 3 | 12 | 5 | BTF3 homologue | M90355_A | 4.15 | 4.07 | undetectable | 1627 | 338 |
| 4 | 4 | 2 | CBFA1/OSF2 | AF053949_B | 0.52 | 0.51 | undetectable | −136 | 14 |
| 2 | 2 | 5 | CDP | M74099_B | 0.45 | 0.44 | undetectable | −54 | 173 |
| 11 | 10 | 5 | cyclin D1 | AML 12 | 1.75 | 1.72 | undetectable | 4205 | 2401 |
| 6 | 6 | 4 | EN2 | L12700_B | 2.93 | 2.88 | undetectable | 1517 | 476 |
| 8 | 15 | 6 | GAPDH | S6-1 | 1.37 | 1.35 | undetectable | 2104 | 1553 |
| 2 | 10 | 2 | GTF2IP1 | AF036613_B | 0.49 | 0.48 | undetectable | −106 | 21 |
| 5 | 12 | 1 | ZRP-1 | AF000974_A | 2.99 | 2.93 | undetectable | 4235 | 1405 |

Hybridization with 5 ug of probe 2 as competitor
Ratio of median value

| | | | | | Not | | | | |
|---|---|---|---|---|---|---|---|---|---|
| | | | | | normalized | beta-actin | 18S | F635 | F532 |
| Block | Column | Row | Gene Name | Probe name | — | 1.20 | 1.11 | median | median |
| 8 | 1 | 5 | 18S | probe 1 | 0.73 | 0.61 | 0.66 | 5617 | 7877 |
| 1 | 11 | 6 | 18S | probe 1 | 0.77 | 0.65 | 0.70 | 50642 | 65367 |
| 1 | 12 | 6 | 18S | probe 1 | 0.68 | 0.56 | 0.61 | 28798 | 42677 |
| 8 | 2 | 5 | 18S | probe 1 | 0.79 | 0.66 | 0.71 | 4808 | 6252 |
| 10 | 1 | 6 | 18S | probe 2 | 1.19 | 0.99 | 1.07 | 1446 | 1275 |
| 10 | 2 | 6 | 18S | probe 2 | 1.24 | 1.03 | 1.12 | 1437 | 1211 |
| 3 | 12 | 6 | 18S | probe 2 | 1.01 | 0.84 | 0.92 | 2904 | 2973 |
| 3 | 11 | 6 | 18S | probe 2 | 0.99 | 0.82 | 0.89 | 2970 | 3112 |
| 5 | 7 | 6 | Beta actin | actin 1 | 0.78 | 0.65 | 0.71 | 2778 | 3771 |
| 5 | 8 | 6 | Beta actin | actin 1 | 0.81 | 0.67 | 0.73 | 2813 | 3723 |
| 10 | 3 | 1 | Beta actin | actin 1 | 0.89 | 0.74 | 0.80 | 2114 | 2491 |
| 10 | 4 | 1 | Beta actin | actin 1 | 0.95 | 0.60 | 0.86 | 1958 | 2142 |
| 4 | 3 | 1 | Beta actin | actin 1 | 0.79 | 0.66 | 0.72 | 886 | 1246 |
| 11 | 14 | 5 | Beta actin | actin 1 | 0.85 | 0.70 | 0.76 | 4081 | 4908 |
| 11 | 13 | 5 | Beta actin | actin 1 | 0.82 | 0.68 | 0.74 | 4163 | 5178 |
| 4 | 4 | 1 | Beta actin | actin 1 | 0.84 | 0.70 | 0.76 | 630 | 861 |
| 6 | 1 | 1 | Beta actin | actin 2 | 1.34 | 1.12 | 1.21 | 6216 | 6179 |
| 3 | 2 | 1 | Beta actin | actin 2 | 1.13 | 0.94 | 1.02 | 2734 | 2573 |
| 4 | 2 | 1 | Beta actin | actin 2 | 1.07 | 0.89 | 0.97 | 3255 | 3107 |
| 6 | 2 | 1 | Beta actin | actin 2 | 1.29 | 1.07 | 1.16 | 5016 | 3954 |
| 1 | 8 | 6 | Beta actin | actin 2 | 0.90 | 0.75 | 0.81 | 5528 | 6304 |
| 4 | 1 | 1 | Beta actin | actin 2 | 0.86 | 0.72 | 0.78 | 3905 | 4676 |
| 3 | 1 | 1 | Beta actin | actin 2 | 1.13 | 0.95 | 1.02 | 6154 | 5479 |
| 5 | 1 | 1 | Beta actin | actin 2 | 0.97 | 0.81 | 0.88 | 2991 | 3266 |
| 5 | 2 | 1 | Beta actin | actin 2 | 0.80 | 0.67 | 0.72 | 1924 | 2563 |
| 1 | 7 | 6 | Beta actin | actin 2 | 0.93 | 0.78 | 0.84 | 8491 | 9183 |
| 3 | 8 | 6 | Beta actin | actin 3 | 1.38 | 1.15 | 1.25 | 7582 | 5556 |
| 3 | 7 | 6 | Beta actin | actin 3 | 1.46 | 1.22 | 1.32 | 9368 | 6469 |
| 11 | 2 | 1 | Beta actin | actin 3 | 1.73 | 1.44 | 1.56 | 1674 | 996 |
| 11 | 1 | 1 | Beta actin | actin 3 | 1.83 | 1.53 | 1.66 | 2150 | 1173 |
| 12 | 2 | 1 | Beta actin | actin 3 | 1.31 | 1.09 | 1.18 | 4607 | 3517 |
| 12 | 1 | 1 | Beta actin | actin 3 | 1.28 | 1.07 | 1.16 | 4478 | 3494 |
| 10 | 2 | 1 | Beta actin | actin 3 | 1.18 | 0.99 | 1.07 | 1003 | 920 |
| 9 | 2 | 1 | Beta actin | actin 3 | 1.65 | 1.37 | 1.49 | 7356 | 4461 |
| 10 | 1 | 1 | Beta actin | actin 3 | 1 26 | 1.05 | 1.14 | 5499 | 4379 |
| 8 | 2 | 1 | Beta actin | actin 3 | 1.69 | 1.41 | 1.52 | 1957 | 1167 |
| 8 | 1 | 1 | Beta actin | actin 3 | 1.60 | 1.33 | 1.44 | 1998 | 1288 |
| 9 | 13 | 5 | Beta actin | actin 3 | 1.67 | 1.39 | 1.50 | 4283 | 2609 |
| 9 | 1 | 1 | Beta actin | actin 3 | 1.70 | 1.41 | 1.53 | 8913 | 5248 |
| 9 | 14 | 5 | Beta actin | actin 3 | 1.60 | 1.33 | 1.44 | 2481 | 1579 |
| 2 | 1 | 1 | Beta actin | actin 3 | 1.18 | 0.98 | 1.06 | 986 | 905 |
| 2 | 2 | 1 | Beta actin | actin 3 | 1.13 | 0.94 | 1.02 | 4407 | 3937 |
| 4 | 2 | 4 | 9G8 splicing | L22253_B | 0.98 | 0.82 | 0.89 | 777 | 875 |
| 9 | 14 | 4 | A-Myb | X13294_B | 2.73 | 2.28 | 2.47 | 1228 | 429 |
| 4 | 8 | 4 | ASH1 | L08424_A | 1.49 | 1.24 | 1.35 | 1332 | 908 |
| 3 | 5 | 3 | BTEB | D31716_B | 2.86 | 2.38 | 2.58 | 1565 | 510 |
| 3 | 12 | 5 | BTF3 homologue | M90355_A | 3.47 | 2.89 | 3.13 | 3479 | 1036 |
| 4 | 4 | 2 | CBFA1/OSF2 | AF053949_B | 1.25 | 1.04 | 1.13 | 62 | 99 |
| 2 | 2 | 5 | CDP | M74099_B | 0.79 | 0.66 | 0.72 | 138 | 296 |
| 11 | 10 | 5 | cyclin D1 | AML 12 | 1.60 | 1.33 | 1.45 | 11710 | 7312 |
| 6 | 6 | 4 | EN2 | L12700_B | 3.61 | 3.01 | 3.26 | 1835 | 458 |
| 8 | 15 | 6 | GAPDH | S6-1 | 2.15 | 1.79 | 1.94 | 3462 | 1593 |
| 2 | 10 | 2 | GTF2IP1 | AF036613_B | 0.66 | 0.57 | 0.62 | −45 | 83 |
| 5 | 12 | 1 | ZRP-1 | AF000974_A | 3.24 | 2.70 | 2.92 | 12043 | 3689 |

| | | | | | Hybridization without competitor | | | | |
|---|---|---|---|---|---|---|---|---|---|
| | | | | | Ratio of median value | | | | |
| | | | | | Not | | | | |
| | | | | | normalized | beta-actin | 18 S | F635 | F532 |
| Block | Column | Row | Gene Name | Probe name | — | 0.56 | — | median | median |
| 8 | 1 | 5 | 18S | probe 1 | 1.01 | 1.80 | saturated | 65181 | 65226 |
| 1 | 11 | 6 | 18S | probe 1 | 1.01 | 1.80 | saturated | 65181 | 65226 |
| 1 | 12 | 6 | 18S | probe 1 | 1.01 | 1.80 | saturated | 65160 | 65187 |
| 8 | 2 | 5 | 18S | probe 1 | 1.01 | 1.80 | saturated | 65154 | 65211 |
| 10 | 1 | 6 | 18S | probe 2 | 1.01 | 1.80 | saturated | 65250 | 65274 |
| 10 | 2 | 6 | 18S | probe 2 | 1.01 | 1.80 | saturated | 65250 | 65283 |
| 3 | 12 | 6 | 18S | probe 2 | 1.01 | 1.80 | saturated | 65157 | 65199 |
| 3 | 11 | 6 | 18S | probe 2 | 1.01 | 1.80 | saturated | 65115 | 65168 |
| 5 | 7 | 6 | Beta actin | actin 1 | 0.66 | 1.18 | saturated | 42650 | 65208 |
| 5 | 8 | 6 | Beta actin | actin 1 | 0.60 | 1.07 | saturated | 32564 | 54998 |
| 10 | 3 | 1 | Beta actin | actin 1 | 0.54 | 0.96 | saturated | 31689 | 59418 |
| 10 | 4 | 1 | Beta actin | actin 1 | 0.50 | 0.89 | saturated | 20804 | 42413 |
| 4 | 3 | 1 | Beta actin | actin 1 | 0.52 | 0.93 | saturated | 5227 | 10326 |
| 11 | 14 | 5 | Beta actin | actin 1 | 0.57 | 1.02 | saturated | 5227 | 9416 |
| 11 | 13 | 5 | Beta actin | actin 1 | 0.57 | 1.02 | saturated | 4828 | 8663 |
| 4 | 4 | 1 | Beta actin | actin 1 | 0.47 | 0.85 | saturated | 3316 | 7269 |
| 6 | 1 | 1 | Beta actin | actin 2 | 0.61 | 1.10 | saturated | 12776 | 21111 |
| 3 | 2 | 1 | Beta actin | actin 2 | 0.60 | 1.07 | saturated | 11482 | 19359 |
| 4 | 2 | 1 | Beta actin | actin 2 | 0.56 | 1.00 | saturated | 9879 | 18018 |
| 6 | 2 | 1 | Beta actin | actin 2 | 0.62 | 1.10 | saturated | 8311 | 13731 |
| 1 | 8 | 6 | Beta actin | actin 2 | 0.56 | 1.01 | saturated | 8060 | 14583 |
| 4 | 1 | 1 | Beta actin | actin 2 | 0.50 | 0.89 | saturated | 6632 | 13645 |
| 3 | 1 | 1 | Beta actin | actin 2 | 0.56 | 1.00 | saturated | 5885 | 10732 |
| 5 | 1 | 1 | Beta actin | actin 2 | 0.51 | 0.92 | saturated | 4246 | 8568 |
| 5 | 2 | 1 | Beta actin | actin 2 | 0.50 | 0.89 | saturated | 3917 | 8126 |
| 1 | 7 | 6 | Beta actin | actin 2 | −0.91 | −1.63 | saturated | −206 | −149 |
| 3 | 8 | 6 | Beta actin | actin 3 | 0.72 | 1.28 | saturated | 12612 | 17918 |
| 3 | 7 | 6 | Beta actin | actin 3 | 0.65 | 1.16 | saturated | 10632 | 16662 |
| 11 | 2 | 1 | Beta actin | actin 3 | 0.87 | 1.55 | saturated | 9874 | 11511 |
| 11 | 1 | 1 | Beta actin | actin 3 | 0.93 | 1.66 | saturated | 8951 | 9743 |
| 12 | 2 | 1 | Beta actin | actin 3 | 0.66 | 1.18 | saturated | 7276 | 11204 |
| 12 | 1 | 1 | Beta actin | actin 3 | 0.61 | 1.09 | saturated | 7196 | 11985 |
| 10 | 2 | 1 | Beta actin | actin 3 | 0.54 | 0.97 | saturated | 6401 | 12065 |
| 9 | 2 | 1 | Beta actin | actin 3 | 0.67 | 1.20 | saturated | 5666 | 8611 |
| 10 | 1 | 1 | Beta actin | actin 3 | 0.53 | 0.94 | saturated | 5565 | 10881 |
| 8 | 2 | 1 | Beta actin | actin 3 | 0.79 | 1.42 | saturated | 4425 | 5686 |
| 8 | 1 | 1 | Beta actin | actin 3 | 0.66 | 1.19 | saturated | 4266 | 6610 |
| 9 | 13 | 5 | Beta actin | actin 3 | 0.62 | 1.11 | saturated | 3873 | 6437 |
| 9 | 1 | 1 | Beta actin | actin 3 | 0.70 | 1.26 | saturated | 3211 | 4705 |
| 9 | 14 | 5 | Beta actin | actin 3 | 0.62 | 1.10 | saturated | 2984 | 5021 |
| 2 | 1 | 1 | Beta actin | actin 3 | 0.48 | 0.86 | saturated | 2319 | 5083 |
| 2 | 2 | 1 | Beta actin | actin 3 | 0.44 | 0.79 | saturated | 2317 | 5572 |
| 4 | 2 | 4 | 9G8 splicing | L22253_B | 0.48 | 0.86 | saturated | 1217 | 2852 |
| 9 | 14 | 4 | A-Myb | X13294_B | 0.84 | 1.50 | saturated | 664 | 877 |
| 4 | 8 | 4 | ASH1 | L08424_A | 0.70 | 1.25 | saturated | 3104 | 4657 |
| 3 | 5 | 3 | BTEB | D31716_B | 1.18 | 2.10 | saturated | 3709 | 3172 |
| 3 | 12 | 5 | BTF3 homologue | M90355_A | 1.14 | 2.03 | saturated | 5707 | 5036 |
| 4 | 4 | 2 | CBFA1/OSF2 | AF053949_B | 0.51 | 0.91 | saturated | 219 | 754 |
| 2 | 2 | 5 | CDP | M74099_B | 0.32 | 0.57 | saturated | 88 | 743 |
| 11 | 10 | 5 | cyclin D1 | AML 12 | 1.03 | 1.84 | saturated | 15590 | 15215 |
| 6 | 6 | 4 | EN2 | L12700_B | 1.41 | 2.51 | saturated | 7429 | 5251 |
| 8 | 15 | 6 | GAPDH | S6-1 | 0.43 | 0.76 | saturated | 3632 | 9331 |
| 2 | 10 | 2 | GTF2IP1 | AF036613_B | 0.36 | 0.65 | saturated | 20 | 458 |
| 5 | 12 | 1 | ZRP-1 | AF000974_A | 1.61 | 2.88 | saturated | 13359 | 8293 |

Appendix 1: Signal normalization using 18S RNA as an internal standard. Two microarray analyses were performed independently, each one comparing the expression of many transcription factors in Jurkat cells and in Jurkat cells treated with the phorbol ester TPA. The signals obtained in the latter case were divided by the signals obtained in the former case to get a ratio of induction by TPA in these cells. The signals were normalized using 18S RNA as a standard (see columns 3 and 4). Since 18S RNA is used as a control in both experiments and that the same type of cells were used, presumably giving very similar results, the ratio of the results obtained in each experiment should be nearing 1. That ratio is presented in column 5.

| | | Column 3 | | |
|---|---|---|---|---|
| | | Jurkat/Jurkat | Column 4 | Column 5 |
| | Column 2 | TPA | Jurkat/Jurkat | Ratio of |
| Column 1 | Accession | ratio | TPA ratio | experiments |
| Gene name | number | experiment 1 | experiment 2 | 1 and 2 |
| 9G8 splicing factor | L22253 | 0.84 | 1.00 | 0.836078512 |
| 9G8 splicing factor | L22253 | 0.77 | 0.99 | 0.779340183 |
| A-Myb | X66087 | 1.32 | 1.38 | 0.950679679 |
| A-Myb | X66087 | 1.34 | 1.43 | 0.937305665 |
| A-Myb | X13294 | 1.12 | 1.21 | 0.924150275 |
| A-Myb | X13294 | 1.12 | 1.21 | 0.924083463 |
| ABF-1 | AF060154 | 0.45 | 0.39 | 1.166895465 |
| ABF-1 | AF060154 | 0.39 | 0.38 | 1.029207795 |
| ABH | NM_006020 | 0.91 | 1.05 | 0.865303363 |
| ABH | NM_006020 | 0.81 | 0.98 | 0.822950019 |
| ABP/ZF | U82613 | 1.32 | 1.64 | 0.804108596 |
| ABP/ZF | U82613 | 1.25 | 1.60 | 0.783304597 |
| AF10 | NM_004641 | 1.24 | 1.31 | 0.947593818 |
| AF10 | NM_004641 | 1.23 | 1.32 | 0.931357689 |
| AIB3 | AF208227 | 1.33 | 1.28 | 1.034779297 |
| AIB3 | NM_014071 | 1.09 | 1.25 | 0.870698314 |
| AIB3 | NM_014071 | 1.07 | 1.36 | 0.784035932 |
| AIB3 | AF208227 | 1.10 | 1.40 | 0.782294079 |
| ALL-1 | U04737 | 1.65 | 1.88 | 0.880126672 |
| ALL-1 | U04737 | 1.58 | 1.88 | 0.838592996 |
| ALL-1 | L04284 | 0.66 | 0.79 | 0.838134698 |
| AML2 | Z35278 | 0.44 | 0.51 | 0.858684813 |
| AML2 | Z35278 | 0.42 | 0.55 | 0.77112205 |
| AML3 | AF001450 | 1.28 | 1.32 | 0.974983445 |
| AML3 | AF001450 | 1.34 | 1.39 | 0.966458433 |
| AP-2gamma | U85658 | 2.57 | 2.62 | 0.978390776 |
| AP-2gamma | U85658 | 2.23 | 2.59 | 0.86381938 |
| AP-4 | X57435 | 1.21 | 1.23 | 0.984438472 |
| AP-4 | X57435 | 1.17 | 1.28 | 0.91144528 |
| AP4 | NM_014374 | 1.39 | 1.59 | 0.871879245 |
| AP4 | NM_014374 | 1.32 | 1.59 | 0.831996755 |
| APBB1 | NM_001164 | 0.95 | 0.97 | 0.984113563 |
| APBB1 | NM_001164 | 0.79 | 0.99 | 0.801180869 |
| APC | M74088 | 1.50 | 1.31 | 1.148676257 |
| APC | M74088 | 1.29 | 1.46 | 0.8859936 |
| APECED | AB006682 | 1.49 | 1.56 | 0.957659838 |
| APECED | AB006682 | 1.38 | 1.65 | 0.837168643 |
| APEX | NM_001641 | 0.88 | 1.13 | 0.783250131 |
| APEX | NM_001641 | 0.84 | 1.08 | 0.780343345 |
| APOBEC2 | NM_006789 | 1.15 | 1.12 | 1.031439776 |
| APOBEC2 | NM_006789 | 1.04 | 1.05 | 0.990111417 |
| APPL | NM_012096 | 1.32 | 1.54 | 0.856820461 |
| APPL | NM_012096 | 1.31 | 1.56 | 0.839878811 |
| AR | NM_000044 | 1.74 | 2.04 | 0.855879355 |
| AR | NM_000044 | 1.60 | 2.01 | 0.796494966 |
| ARNT | M69238 | 1.25 | 1.42 | 0.880056649 |
| ARNT | M69238 | 1.24 | 1.42 | 0.876705905 |
| ARNT | Y18500 | 0.78 | 0.96 | 0.816130578 |
| ASH2L2 | AF056717 | 1.34 | 1.35 | 0.994678817 |
| ASH2L2 | AF056717 | 1.38 | 1.40 | 0.991252318 |
| ATBF1 | NM_006885 | 0.90 | 1.01 | 0.889758762 |
| ATBF1 | NM_006885 | 0.90 | 1.02 | 0.879456944 |
| ATF | D90209 | 1.05 | 1.01 | 1.035713928 |
| ATF | D90209 | 0.97 | 1.01 | 0.960323304 |
| ATF-a | X52943 | 1.54 | 1.88 | 0.817277421 |
| ATF-a | X52943 | 1.51 | 1.93 | 0.780957523 |
| ATF1 | NM_005171 | 0.84 | 0.91 | 0.927916867 |
| ATF1 | NM_005171 | 0.87 | 1.02 | 0.854281302 |
| ATF6 | NM_007348 | 1.29 | 1.29 | 1.00327664 |
| ATF6 | NM_007348 | 1.09 | 1.28 | 0.856533977 |
| BACH1 | NM_001186 | 1.49 | 1.31 | 1.137064444 |
| BACH1 | NM_001186 | 1.45 | 1.62 | 0.891057108 |
| BAPX1 | NM_001189 | 2.55 | 2.33 | 1.093826453 |
| BAPX1 | NM_001189 | 2.46 | 2.59 | 0.946872482 |
| BARX2 | NM_003658 | 1.17 | 1.27 | 0.917084438 |
| BARX2 | NM_003658 | 1.14 | 1.37 | 0.830998058 |
| BCL2 | NM_000633 | 1.43 | 1.65 | 0.866945304 |
| BCL2 | NM_000633 | 1.37 | 1.70 | 0.806442848 |
| BCL3 | U05822 | 1.11 | 1.26 | 0.877431885 |
| BCL3 | M31732 | 1.17 | 1.38 | 0.848343893 |
| BCL3 | M31732 | 1.13 | 1.37 | 0.825031918 |
| BCL3 | U05822 | 1.02 | 1.30 | 0.790257156 |
| Beta-actin | X00351 | 1.02 | 1.19 | 0.855958172 |
| Beta-actin | X00351 | 1.02 | 1.21 | 0.843968769 |
| Beta-actin | X00351 | 1.01 | 1.21 | 0.837209294 |
| Beta-actin | X00351 | 1.00 | 1.19 | 0.836410947 |
| beta-catenin | X89593 | 2.01 | 2.06 | 0.977986591 |
| beta-catenin | X89593 | 1.99 | 2.11 | 0.942592932 |
| BF-2 | X74143 | 1.28 | 1.38 | 0.931388014 |
| BF-2 | X74143 | 1.22 | 1.37 | 0.894927517 |
| BFP/ZNF179 | AB026054 | 1.33 | 1.32 | 1.005754548 |
| BFP/ZNF179 | AB026054 | 1.36 | 1.37 | 0.993222418 |
| BIRC4 | NM_001167 | 1.51 | 1.44 | 1.054435009 |
| BIRC4 | NM_001167 | 1.40 | 1.50 | 0.932289706 |
| BMZF3 | NM_005773 | 0.92 | 1.08 | 0.850837495 |
| BMZF3 | NM_005773 | 0.90 | 1.13 | 0.798215326 |
| brahma | X72889 | 5.90 | 5.49 | 1.074544412 |
| brahma | X72889 | 5.14 | 5.97 | 0.86166573 |
| BRCA2 | NM_000059 | 1.45 | 1.75 | 0.824507422 |
| BRCA2 | NM_000059 | 1.39 | 1.74 | 0.798236353 |
| Brn-3B | U06233 | 1.48 | 1.37 | 1.078166711 |
| Brn-3B | U06233 | 1.47 | 1.50 | 0.974841891 |
| Brn-4 | X82324 | 1.57 | 1.06 | 1.486851514 |
| Brn-4 | X82324 | 1.29 | 1.07 | 1.198217087 |
| BRS3 | NM_001727 | 2.71 | 2.75 | 0.983814035 |
| BRS3 | NM_001727 | 2.36 | 2.77 | 0.851828571 |
| BTEB | D31716 | 4.86 | 4.21 | 1.153934489 |
| BTEB | D31716 | 4.30 | 4.32 | 0.995197771 |
| BTEB2 | D14520 | 1.25 | 1.27 | 0.978590601 |
| BTEB2 | D14520 | 1.30 | 1.39 | 0.933625786 |
| BTF3 | NM_001207 | 1.05 | 1.10 | 0.955111894 |
| BTF3 | NM_001207 | 0.99 | 1.08 | 0.913787418 |
| BTF3a | M90352 | 2.83 | 2.32 | 1.219855319 |
| BTF3a | M90352 | 2.70 | 2.39 | 1.130461687 |
| BTF3L1 | NM_001208 | 1.22 | 1.07 | 1.137813523 |
| BTF3L1 | NM_001208 | 1.16 | 1.05 | 1.102860167 |
| BTF3L3 | M90356 | 1.44 | 1.37 | 1.049188317 |
| BTF3L3 | M90356 | 1.24 | 1.34 | 0.927268611 |
| bZip protein B-ATF | U15460 | 1.07 | 1.14 | 0.9426678 |
| bZip protein B-ATF | U15460 | 0.97 | 1.08 | 0.901877866 |
| c-Ets-1 | X14798 | 1.09 | 1.25 | 0.873492353 |
| c-Ets-1 | X14798 | 1.10 | 1.32 | 0.830363686 |
| c-maf | AF055376 | 5.74 | 4.79 | 1.19705637 |
| c-maf | AF055376 | 4.91 | 5.10 | 0.962031195 |
| c-Rel | M11595 | 1.33 | 1.41 | 0.946493027 |
| c-Rel | X75042 | 1.32 | 1.46 | 0.902036285 |
| c-Rel | M11595 | 1.27 | 1.42 | 0.889929469 |
| c-Rel | X75042 | 1.14 | 1.47 | 0.777782886 |
| C2H2 ZNF | AF033199 | 1.07 | 1.14 | 0.938338671 |
| C2H2 ZNF | AF033199 | 0.99 | 1.16 | 0.852890579 |
| C2H2-type ZNF | U95991 | 1.19 | 1.01 | 1.173282928 |
| C2H2-type ZNF | U95991 | 0.98 | 1.04 | 0.942590144 |
| C2ORF3 | NM_003203 | 1.46 | 1.22 | 1.196699322 |
| C2ORF3 | NM_003203 | 1.01 | 0.93 | 1.093811577 |
| CBF (5) | M37197 | 4.06 | 4.25 | 0.956014195 |
| CBF (5) | M37197 | 3.60 | 4.09 | 0.88090602 |
| CBF1 | AF098297 | 1.61 | 1.63 | 0.991664197 |
| CBF1 | AF098297 | 1.38 | 1.78 | 0.772546908 |
| CBFA1 | L40992 | 1.30 | 1.45 | 0.898057655 |
| CBFA1 | L40992 | 1.26 | 1.46 | 0.865127809 |
| CBFA1/OSF2 | AF053949 | 1.22 | 1.28 | 0.951727989 |
| CBFA1/OSF2 | AF053949 | 1.22 | 1.33 | 0.92146037 |
| CBFA2T1 | NM_004349 | 1.49 | 1.65 | 0.901008111 |
| CBFA2T1 | NM_004349 | 1.24 | 1.59 | 0.780002118 |
| CBFB | L20298 | 2.33 | 2.74 | 0.851333501 |
| CBFB | L20298 | 2.36 | 2.91 | 0.8088749 |
| CDP | M74099 | 1.39 | 1.61 | 0.85914075 |
| CDP | M74099 | 1.27 | 1.64 | 0.77621359 |
| CEBPB | NM_005194 | 1.24 | 1.47 | 0.846246886 |
| CEBPB | NM_005194 | 1.26 | 1.49 | 0.846246188 |
| CEBPD | NM_005195 | 0.83 | 1.00 | 0.829917576 |
| CEBPD | NM_005195 | 0.84 | 1.03 | 0.822579365 |
| CEBPE | U48866 | 1.91 | 2.01 | 0.948532903 |
| CEBPE | U48866 | 2.06 | 2.38 | 0.86669978 |
| CEZANNE | NM_020205 | 2.88 | 2.96 | 0.974633442 |
| CEZANNE | NM_020205 | 2.65 | 2.83 | 0.935357017 |
| CHD1 | NM_001270 | 1.62 | 1.59 | 1.014951939 |
| CHD1 | NM_001270 | 1.43 | 1.59 | 0.898362477 |
| CHD4 | NM_001273 | 1.54 | 1.70 | 0.909055986 |
| CHD4 | NM_001273 | 1.49 | 1.72 | 0.862018232 |
| CHFR | NM_018223 | 4.35 | 4.43 | 0.982194772 |
| CHFR | NM_018223 | 3.92 | 4.36 | 0.899117503 |
| CHN1 | NM_001822 | 1.42 | 1.53 | 0.927629676 |
| CHN1 | NM_001822 | 1.37 | 1.49 | 0.923095091 |
| CIS4 | NM_004232 | 1.67 | 1.79 | 0.935688257 |
| CIS4 | NM_004232 | 1.82 | 2.13 | 0.851569476 |
| CITED1 | NM_004143 | 1.10 | 1.30 | 0.850853943 |
| CITED1 | NM_004143 | 1.17 | 1.39 | 0.844249881 |
| CNBP | M28372 | 0.67 | 0.54 | 1.233592517 |
| CNBP | M28372 | 0.62 | 0.54 | 1.163359863 |
| coactivator EBV nuclear | U22055 | 0.82 | 0.94 | 0.869546763 |
| protein 2 | | | | |
| coactivator EBV nuclear | U22055 | 0.81 | 1.00 | 0.810099254 |
| protein 2 | | | | |
| COPEB | NM_001300 | 1.14 | 1.29 | 0.885046712 |
| COPEB | NM_001300 | 1.12 | 1.34 | 0.833843243 |
| COPS5 | NM_006837 | 2.46 | 2.14 | 1.148421053 |
| COPS5 | NM_006837 | 2.48 | 2.32 | 1.071355007 |
| CP2 | U01965 | 1.01 | 1.23 | 0.82004865 |
| CP2 | U01965 | 1.00 | 1.30 | 0.771414141 |
| CR53 | AF017433 | 1.33 | 1.33 | 0.997732351 |
| CR53 | AF017433 | 1.29 | 1.39 | 0.925956448 |
| CRE-BP1 | J05623 | 1.13 | 1.38 | 0.819277436 |
| CRE-BP1 | J05623 | 1.02 | 1.26 | 0.815059942 |
| CREB | M27691 | 0.92 | 1.09 | 0.842697518 |
| CREB | M27691 | 0.85 | 1.06 | 0.7964146 |
| CREBBP | NM_004380 | 1.09 | 1.25 | 0.872661186 |
| CREBBP | NM_004380 | 1.12 | 1.30 | 0.86705145 |
| CREBPA | NM_004904 | 1.26 | 1.30 | 0.971711147 |
| CREBPA | NM_004904 | 1.10 | 1.24 | 0.887551154 |
| CROC4 | NM_006365 | 1.15 | 1.25 | 0.926055212 |
| CROC4 | NM_006365 | 1.16 | 1.38 | 0.842320854 |
| CRSP70 | NM_004831 | 0.91 | 1.06 | 0.854668195 |
| CRSP70 | NM_004831 | 0.92 | 1.15 | 0.803384327 |
| CRSP9 | NM_004270 | 1.37 | 1.49 | 0.919973517 |
| CRSP9 | NM_004270 | 1.40 | 1.53 | 0.919262135 |
| CSDA | NM_003651 | 2.00 | 2.09 | 0.956497534 |
| CSDA | NM_003651 | 1.79 | 2.09 | 0.857935728 |
| CSPG4 | NM_001897 | 6.91 | 6.16 | 1.121744511 |
| CSPG4 | NM_001897 | 6.24 | 6.25 | 0.998642122 |
| cyclin T1 | AF048730 | 1.27 | 1.54 | 0.823279433 |
| cyclin T1 | AF048730 | 1.20 | 1.47 | 0.813677962 |
| cyclin T2a | AF048731 | 1.50 | 1.54 | 0.973727374 |
| cyclin T2a | AF048731 | 1.65 | 1.70 | 0.971786333 |
| Daxx | AB015051 | 1.22 | 1.49 | 0.814149894 |
| Daxx | AB015051 | 1.16 | 1.45 | 0.796739358 |
| DB1 | D28118 | 1.21 | 1.38 | 0.873780256 |
| DB1 | D28118 | 1.20 | 1.38 | 0.871224304 |
| DDXBP1 | NM_016166 | 1.20 | 1.32 | 0.908250709 |
| DDXBP1 | NM_016166 | 1.14 | 1.32 | 0.865664426 |
| DED | AJ249940 | 0.85 | 0.90 | 0.947823489 |
| DED | AJ249940 | 0.84 | 0.90 | 0.93599742 |
| DEK | S89712 | 1.38 | 1.62 | 0.856330516 |
| DEK | S89712 | 1.32 | 1.55 | 0.852478465 |
| DFFB | NM_004402 | 1.36 | 1.40 | 0.968276574 |
| DFFB | NM_004402 | 1.22 | 1.55 | 0.787420865 |
| DIP1 | NM_012142 | 1.39 | 1.14 | 1.217929208 |
| DIP1 | NM_012142 | 1.17 | 1.15 | 1.01617335 |
| DLC1 | NM_006094 | 3.06 | 3.29 | 0.931248269 |
| DLC1 | NM_006094 | 2.97 | 3.29 | 0.903164687 |
| DLX3 | NM_005220 | 1.13 | 1.26 | 0.894141987 |
| DLX5 | NM_005221 | 1.45 | 1.39 | 1.04166642 |
| DLX5 | NM_005221 | 1.25 | 1.61 | 0.775477519 |
| DMAHP | X84813 | 1.10 | 1.29 | 0.851587242 |
| DMAHP | X84813 | 1.08 | 1.31 | 0.825399746 |
| DMRT1 | AJ276801 | 1.41 | 1.41 | 1.002793104 |
| DMRT1 | AJ276801 | 1.43 | 1.48 | 0.961743556 |
| DNA-binding protein | X60824 | 1.36 | 1.52 | 0.897844438 |
| DNA-binding protein | X60824 | 1.32 | 1.48 | 0.88927803 |
| DNASE1 | NM_005223 | 1.21 | 1.25 | 0.964151008 |
| DNASE1 | NM_005223 | 0.97 | 1.21 | 0.798481304 |
| DNASE2 | NM_001375 | 2.98 | 3.43 | 0.867988126 |
| DNASE2 | NM_001375 | 2.89 | 3.55 | 0.815129956 |
| DRA | NM_000111 | 1.26 | 1.39 | 0.904139999 |
| DRA | NM_000111 | 1.21 | 1.41 | 0.862444488 |
| DREAM | AJ131730 | 0.78 | 0.96 | 0.819901761 |
| DREAM | AJ131730 | 0.76 | 0.98 | 0.770874238 |
| E2F1 | M96577 | 0.89 | 1.03 | 0.869321414 |
| E2F1 | M96577 | 0.91 | 1.05 | 0.867695906 |
| EAR-1r | D16815 | 2.06 | 2.10 | 0.984212792 |
| EAR-1r | D16815 | 1.88 | 2.21 | 0.850783292 |
| EGR1 | X52541 | 1.47 | 1.50 | 0.979883348 |
| EGR1 | X52541 | 1.44 | 1.51 | 0.953589751 |
| EGR1 | M17254 | 0.86 | 1.03 | 0.832083695 |
| EGR1 | M17254 | 0.87 | 1.05 | 0.827505943 |
| EGR4 | NM_001965 | 0.60 | 0.71 | 0.840382873 |
| EGR4 | NM_001965 | 0.63 | 0.81 | 0.775954581 |
| EKLF | U65404 | 0.98 | 1.04 | 0.944031465 |
| EKLF | U65404 | 0.96 | 1.03 | 0.935317019 |
| ELF1 | M82882 | 1.76 | 1.83 | 0.964878433 |
| ELF1 | M82882 | 1.62 | 1.76 | 0.921751518 |
| ELF4 | NM_001421 | 1.45 | 1.41 | 1.027947336 |
| ELF4 | NM_001421 | 1.36 | 1.37 | 0.991044834 |
| ELK3 | NM_005230 | 1.28 | 1.57 | 0.815739725 |
| ELK3 | NM_005230 | 1.33 | 1.68 | 0.790796088 |
| ELL | NM_006532 | 0.95 | 1.16 | 0.822566492 |
| ELL | NM_006532 | 0.95 | 1.16 | 0.819455294 |
| elongation factor 1- | X16869 | 1.35 | 1.50 | 0.8947725 |
| alpha | | | | |
| elongation factor 1- | X16869 | 1.36 | 1.59 | 0.853485168 |
| alpha | | | | |
| | L34587 | 1.41 | 1.64 | 0.861800291 |
| elongation factor SIII | | | | |
| elongation factor SIII | L34587 | 1.49 | 1.82 | 0.820065033 |
| elongation factor-1- | Z21507 | 0.81 | 0.99 | 0.81190776 |
| delta | | | | |
| elongation factor-1- | Z21507 | 0.78 | 1.00 | 0.782148893 |
| delta | | | | |
| EN1 | L12698 | 1.36 | 1.45 | 0.935865444 |
| EN1 | L12698 | 1.23 | 1.47 | 0.836794344 |
| EPAS1 | NM_001430 | 1.18 | 1.38 | 0.856844874 |
| EPAS1 | NM_001430 | 1.15 | 1.46 | 0.783761416 |
| ERCC2 | X52222 | 5.72 | 4.80 | 1.193231705 |
| ERCC2 | X52222 | 5.33 | 4.73 | 1.127089247 |
| ERCC3 | NM_000122 | 1.36 | 1.57 | 0.863467286 |
| ERCC3 | NM_000122 | 1.30 | 1.60 | 0.812147676 |
| ERF-2 | X78992 | 2.14 | 2.41 | 0.889330713 |
| ERF-2 | X78992 | 2.26 | 2.55 | 0.883602051 |
| ERG | NM_004449 | 1.62 | 1.42 | 1.142428678 |
| ERG | NM_004449 | 1.49 | 1.50 | 0.996969892 |
| ERM | X96375 | 4.16 | 4.29 | 0.969559654 |
| ERM | X96375 | 3.27 | 3.55 | 0.921520209 |
| ERT | AF017307 | 2.43 | 2.68 | 0.90894817 |
| ERT | AF017307 | 2.51 | 2.82 | 0.891141057 |
| ESRRG | NM_001438 | 0.95 | 1.13 | 0.839582135 |
| ESRRG | NM_001438 | 0.95 | 1.15 | 0.821231854 |
| ETR101 | NM_004907 | 2.74 | 2.75 | 0.997375352 |
| ETR101 | NM_004907 | 2.49 | 2.80 | 0.887790293 |
| Ets transcription factor | AF115403 | 1.14 | 1.31 | 0.87442124 |
| ESE-2b | | | | |
| Ets transcription factor | AF115403 | 1.11 | 1.43 | 0.77156259 |
| ESE-2b | | | | |
| Ets-1 gene | AF193068 | 1.21 | 1.38 | 0.874625305 |
| Ets-1 gene | AF193068 | 1.22 | 1.40 | 0.868962372 |
| Ets-like | U30174 | 1.40 | 1.23 | 1.131217765 |
| Ets-like | U30174 | 1.49 | 1.35 | 1.098811633 |
| Ets-like | Z49980 | 1.61 | 1.51 | 1.067048232 |
| Ets-like | Z49980 | 1.54 | 1.56 | 0.991710772 |
| Ets2 | M30137 | 1.75 | 2.02 | 0.86945137 |
| Ets2 | M30137 | 1.78 | 2.11 | 0.844919404 |
| ETV1 | NM_004956 | 1.13 | 1.25 | 0.910122678 |
| ETV1 | NM_004956 | 1.39 | 1.59 | 0.871215971 |
| ETV6 | U45432 | 1.38 | 1.43 | 0.965065589 |
| ETV6 | NM_001987 | 0.90 | 1.11 | 0.811726255 |
| Evi-1 | S82592 | 2.53 | 2.10 | 1.208239627 |
| Evi-1 | S82592 | 2.26 | 2.15 | 1.055074375 |
| EWSR1 | NM_005243 | 1.01 | 1.28 | 0.789906804 |
| EWSR1 | NM_005243 | 1.00 | 1.28 | 0.783731221 |
| EZH2 | U61145 | 1.26 | 1.35 | 0.932953273 |
| EZH2 | U61145 | 1.27 | 1.39 | 0.907474288 |
| FACTP140 | NM_007192 | 1.43 | 1.48 | 0.96265369 |
| FACTP140 | NM_007192 | 1.41 | 1.48 | 0.954817504 |
| Fas-binding protein | AF015956 | 0.90 | 1.08 | 0.833884369 |
| Daxx | | | | |
| Fas-binding protein | AF015956 | 0.89 | 1.09 | 0.81465638 |
| Daxx | | | | |
| FBW1A | AF129530 | 1.31 | 1.45 | 0.900471742 |
| FBW1A | AF129530 | 1.27 | 1.54 | 0.829306514 |
| FGD1 | U11690 | 1.33 | 1.14 | 1.173441119 |
| FGD1 | U11690 | 1.21 | 1.23 | 0.990554056 |
| FGR | NM_005248 | 1.33 | 1.58 | 0.839283541 |
| FGR | NM_005248 | 1.27 | 1.60 | 0.790883893 |
| FHL1 | AF110763 | 1.56 | 1.77 | 0.88200997 |
| FHL1 | AF110763 | 1.45 | 1.76 | 0.822210318 |
| FKHL7 | AF048693 | 3.42 | 3.29 | 1.040543697 |
| FKHL7 | AF048693 | 3.65 | 3.62 | 1.006927826 |
| FKHR | AF032885 | 2.42 | 2.08 | 1.161966778 |
| FKHR | AF032885 | 2.36 | 2.18 | 1.082816723 |
| FKHRL1P1 | AF032887 | 1.42 | 1.54 | 0.924383924 |
| FKHRL1P1 | AF032887 | 1.46 | 1.60 | 0.912174436 |
| FLI_CDNA | AL360183 | 1.33 | 1.28 | 1.036167415 |
| FLI_CDNA | AL360183 | 1.37 | 1.37 | 0.996443864 |
| FLJ10173 | NM_018014 | 1.04 | 1.04 | 0.999229429 |
| FLJ10173 | NM_018014 | 1.01 | 1.01 | 0.996944727 |
| FLJ10251 | NM_018039 | 1.31 | 1.43 | 0.911977997 |
| FLJ10251 | NM_018039 | 1.31 | 1.46 | 0.897214657 |
| FLJ10339 | NM_018063 | 1.62 | 1.87 | 0.866263178 |
| FLJ10339 | NM_018063 | 1.53 | 1.86 | 0.822236451 |
| FLJ10469 | NM_018102 | 0.94 | 1.09 | 0.865336872 |
| FLJ10469 | NM_018102 | 0.94 | 1.09 | 0.865236102 |
| FLJ10688 | AK001550 | 0.97 | 1.11 | 0.881574929 |
| FLJ10688 | AK001550 | 0.95 | 1.19 | 0.802514491 |
| FLJ10891 | NM_018260 | 1.22 | 1.31 | 0.928051813 |
| FLJ10891 | NM_018260 | 1.15 | 1.38 | 0.83802715 |
| FLJ10909 | AK001771 | 2.36 | 2.38 | 0.988865574 |
| FLJ10909 | AK001771 | 2.19 | 2.39 | 0.917728349 |
| FLJ11015 | NM_018300 | 1.10 | 1.19 | 0.922727928 |
| FLJ11015 | NM_018300 | 0.99 | 1.17 | 0.845365497 |
| FLJ11137 | NM_018337 | 1.43 | 1.64 | 0.875337744 |
| FLJ11137 | NM_018337 | 1.33 | 1.60 | 0.831187459 |
| FLJ11340 | AK002202 | 3.74 | 3.96 | 0.944037815 |
| FLJ11340 | AK002202 | 3.72 | 4.02 | 0.925252175 |
| FLJ11344 | AK002206 | 1.11 | 1.24 | 0.900786005 |
| FLJ11344 | AK002206 | 1.22 | 1.39 | 0.879708376 |
| FLJ11688 | AK021750 | 1.31 | 1.41 | 0.925531252 |
| FLJ11688 | AK021750 | 1.35 | 1.50 | 0.901037884 |
| FLJ12606 | AK022668 | 1.11 | 1.15 | 0.965529216 |
| FLJ12606 | AK022668 | 0.96 | 1.10 | 0.876202729 |
| FLJ12628 | AK022690 | 1.30 | 1.38 | 0.938935506 |
| FLJ12628 | AK022690 | 1.25 | 1.36 | 0.925116959 |
| FLJ12644 | AK000909 | 0.98 | 1.09 | 0.901825689 |
| FLJ12644 | AK000909 | 1.02 | 1.16 | 0.874104763 |
| FLJ13479 | AK023541 | 1.12 | 1.35 | 0.830838509 |
| FLJ13479 | AK023541 | 1.05 | 1.27 | 0.825925564 |
| FLJ20337 | NM_017772 | 1.55 | 1.60 | 0.969110576 |
| FLJ20337 | NM_017772 | 1.60 | 1.66 | 0.966477023 |
| FLJ20428 | AK000435 | 1.00 | 1.13 | 0.88699187 |
| FLJ20428 | AK000435 | 1.03 | 1.16 | 0.883146291 |
| FLJ20438 | ak000445 | 2.61 | 2.97 | 0.876697181 |
| FLJ20438 | ak000445 | 2.41 | 2.99 | 0.807812353 |
| FLJ22332 | AK025985 | 1.37 | 1.67 | 0.823105199 |
| FLJ22332 | AK025985 | 1.22 | 1.52 | 0.80219778 |
| FLJ22973 | AK026626 | 0.93 | 1.11 | 0.841359026 |
| FLJ22973 | AK026626 | 0.94 | 1.14 | 0.825377156 |
| FOG2 | NM_012082 | 1.03 | 1.10 | 0.930301277 |
| FOG2 | NM_012082 | 1.11 | 1.24 | 0.901732208 |
| FOSL2 | NM_005253 | 1.42 | 1.73 | 0.818161857 |
| FOSL2 | NM_005253 | 1.40 | 1.80 | 0.775807396 |
| FOXD2 | NM_004474 | 1.36 | 1.49 | 0.918399567 |
| FOXD2 | NM_004474 | 1.35 | 1.48 | 0.912187342 |
| FOXD3 | NM_012183 | 1.67 | 1.55 | 1.072311149 |
| FOXD3 | NM_012183 | 1.55 | 1.63 | 0.952188792 |
| FOXO3A | NM_001455 | 0.87 | 1.10 | 0.789948212 |
| FOXO3A | NM_001455 | 0.85 | 1.09 | 0.780082817 |
| FRA-1 | X16707 | 1.19 | 1.22 | 0.975373174 |
| FRA-1 | X16707 | 1.15 | 1.25 | 0.920368654 |
| FREAC1 | U13219 | 1.33 | 1.44 | 0.920850002 |
| FREAC1 | U13219 | 1.33 | 1.47 | 0.900355759 |
| FREAC10 | AF042831 | 1.37 | 1.53 | 0.895510594 |
| FREAC10 | AF042831 | 1.29 | 1.49 | 0.862685753 |
| FREAC6 | L13203 | 0.68 | 0.76 | 0.894745854 |
| FREAC6 | L13203 | 0.65 | 0.75 | 0.865206105 |
| FREAC7 | U13225 | 0.82 | 0.70 | 1.159351607 |
| FREAC7 | U13225 | 0.70 | 0.72 | 0.971169803 |
| frpHE | AF026692 | 1.02 | 1.11 | 0.917414227 |
| frpHE | AF026692 | 0.99 | 1.17 | 0.852068204 |
| GABPB1 | NM_005254 | 1.74 | 1.77 | 0.98532103 |
| GABPB1 | NM_005254 | 1.74 | 1.82 | 0.956282084 |
| GADD 153 | s40706 | 1.28 | 1.47 | 0.871741003 |
| GADD 153 | s40706 | 1.09 | 1.42 | 0.76973024 |
| GAPDH | M33197 | 0.58 | 0.61 | 0.949162972 |
| GAPDH | M33197 | 0.56 | 0.59 | 0.947048611 |
| GCMA | NM_003643 | 1.20 | 1.32 | 0.908325507 |
| GCMA | NM_003643 | 1.15 | 1.32 | 0.870108289 |
| GCN5L1 | NM_001487 | 0.80 | 0.93 | 0.856784201 |
| GCN5L1 | NM_001487 | 0.73 | 0.90 | 0.820087548 |
| GIOT-1 | AB021641 | 1.29 | 1.45 | 0.884372648 |
| GIOT-1 | AB021641 | 1.22 | 1.50 | 0.812297818 |
| GIOT-2 | NM_016264 | 0.93 | 1.07 | 0.868101488 |
| GIOT-2 | NM_016264 | 0.91 | 1.10 | 0.82720992 |
| GIOT-3 | NM_016265 | 0.87 | 0.97 | 0.893216374 |
| GIOT-3 | NM_016265 | 0.86 | 0.97 | 0.884789805 |
| GIOT-4 | NM_016266 | 1.73 | 2.15 | 0.806073313 |
| GIOT-4 | NM_016266 | 1.78 | 2.27 | 0.783305867 |
| GLI | X07384 | 1.34 | 1.32 | 1.019171414 |
| GLI | X07384 | 1.28 | 1.34 | 0.958829722 |
| GLI3 | M57609 | 2.18 | 1.98 | 1.098071683 |
| GLI3 | M57609 | 1.95 | 2.06 | 0.944192578 |
| GPX5 | NM_001509 | 0.94 | 1.08 | 0.865100605 |
| GPX5 | NM_001509 | 1.37 | 1.62 | 0.847040407 |
| GRLF1 | NM_004491 | 0.79 | 0.87 | 0.906709069 |
| GRLF1 | NM_004491 | 0.71 | 0.80 | 0.885942625 |
| GTF2B | NM_001514 | 2.16 | 2.30 | 0.937394785 |
| GTF2B | NM_001514 | 1.95 | 2.44 | 0.799673306 |
| GTF2E1 | NM_005513 | 0.76 | 0.98 | 0.784112092 |
| GTF2E1 | NM_005513 | 0.74 | 0.96 | 0.769972038 |
| GTF2I | NM_001518 | 2.68 | 2.80 | 0.958900201 |
| GTF2I | NM_001518 | 2.58 | 2.96 | 0.869928806 |
| GTF2IP1 | AF036613 | 0.95 | 0.70 | 1.359169172 |
| GTF2IP1 | AF036613 | 0.84 | 0.84 | 0.988769754 |
| GTF3A | NM_002097 | 1.49 | 1.59 | 0.932083753 |
| GTF3A | NM_002097 | 1.58 | 1.70 | 0.931723008 |
| GTF3C1 | NM_001520 | 2.07 | 2.25 | 0.919747554 |
| GTF3C1 | NM_001520 | 1.99 | 2.34 | 0.847308047 |
| GTF3C2 | NM_001521 | 1.31 | 1.28 | 1.027107124 |
| GTF3C2 | NM_001521 | 1.22 | 1.29 | 0.943844023 |
| GTF3C3 | NM_012086 | 1.64 | 1.64 | 0.996581398 |
| GTF3C3 | NM_012086 | 1.52 | 1.67 | 0.907893639 |
| GTF3C4 | NM_012204 | 1.11 | 1.28 | 0.860437792 |
| GTF3C4 | NM_012204 | 1.13 | 1.33 | 0.851773956 |
| GTP | AF054183 | 1.98 | 2.25 | 0.880760132 |
| GTP | AF054183 | 1.86 | 2.27 | 0.818437867 |
| H1F3 | M60746 | 1.08 | 1.27 | 0.853497342 |
| H1F3 | M60746 | 1.10 | 1.33 | 0.824710104 |
| H2AFX | X14850 | 1.24 | 1.33 | 0.934617922 |
| H2AFX | X14850 | 1.19 | 1.40 | 0.849848259 |
| H4 | X67081 | 0.83 | 0.97 | 0.859373264 |
| H4 | X67081 | 0.84 | 1.00 | 0.842811776 |
| hairless | AF039196 | 1.39 | 1.46 | 0.951801096 |
| hairless | AF039196 | 1.37 | 1.53 | 0.896272718 |
| HAP2 | M59079 | 1.59 | 1.49 | 1.062457371 |
| HAP2 | M59079 | 1.33 | 1.71 | 0.780793131 |
| HAT1 | NM_003642 | 1.05 | 0.86 | 1.223229142 |
| HAT1 | NM_003642 | 1.06 | 1.04 | 1.026464835 |
| HB16 | M31630 | 0.97 | 1.09 | 0.894291244 |
| HB16 | M31630 | 1.01 | 1.26 | 0.797052293 |
| HB9 | U07663 | 2.64 | 2.61 | 1.013508831 |
| HB9 | U07664 | 0.92 | 1.03 | 0.895489189 |
| HBOA | NM_007067 | 1.27 | 1.37 | 0.929935594 |
| HBOA | NM_007067 | 1.23 | 1.35 | 0.912294782 |
| HCF-2 | AF117210 | 1.43 | 1.56 | 0.918694023 |
| HCF-2 | AF117210 | 1.43 | 1.61 | 0.888145397 |
| HD-ZNF1 | NM_004876 | 1.11 | 1.22 | 0.910541692 |
| HD-ZNF1 | NM_004876 | 1.05 | 1.19 | 0.884372648 |
| HDAC1 | NM_004964 | 0.83 | 0.97 | 0.851140233 |
| HDAC1 | NM_004964 | 0.82 | 0.97 | 0.844737874 |
| HDAC4 | NM_006037 | 2.76 | 2.40 | 1.153229661 |
| HDAC4 | NM_006037 | 1.64 | 1.99 | 0.825094678 |
| HEB | M83233 | 0.75 | 0.89 | 0.83812814 |
| HEB | M83233 | 0.73 | 0.90 | 0.814864061 |
| HEN1 | M96739 | 1.51 | 1.61 | 0.937658625 |
| HEN1 | M96739 | 1.49 | 1.69 | 0.883530062 |
| HERP1 | AF232238 | 1.64 | 1.78 | 0.918811847 |
| HERP1 | AF232238 | 1.48 | 1.69 | 0.873182906 |
| HERP2 | AF232239 | 0.88 | 0.97 | 0.913791819 |
| HERP2 | AF232239 | 0.82 | 1.01 | 0.814129508 |
| HES4 | AB048791 | 1.12 | 1.24 | 0.906421263 |
| HES4 | AB048791 | 1.17 | 1.31 | 0.892326717 |
| HGS | NM_004712 | 1.13 | 1.22 | 0.925941974 |
| HGS | NM_004712 | 1.08 | 1.23 | 0.884627755 |
| HIC1 | NM_006497 | 1.01 | 1.19 | 0.84718439 |
| HIC1 | NM_006497 | 0.94 | 1.20 | 0.789513268 |
| HIVEP1 | NM_002114 | 1.24 | 1.14 | 1.08520656 |
| HIVEP1 | NM_002114 | 1.03 | 1.16 | 0.893216374 |
| HIVEP2 | NM_006734 | 2.86 | 2.87 | 0.99372865 |
| HKE4 | NM_006979 | 1.51 | 1.70 | 0.89115193 |
| HKE4 | NM_006979 | 1.35 | 1.66 | 0.814320291 |
| HLF | M95585 | 1.28 | 1.32 | 0.971118298 |
| HLF | M95586 | 1.15 | 1.26 | 0.910803018 |
| HMG-1 | D63874 | 1.27 | 1.25 | 1.015741343 |
| HMG-1 | D63874 | 1.23 | 1.22 | 1.008346304 |
| HMG-2 | X62534 | 1.70 | 1.82 | 0.938295788 |
| HMG-2 | X62534 | 1.55 | 1.76 | 0.878616998 |
| HMG17 | NM_005517 | 0.99 | 1.14 | 0.868604212 |
| HMG17 | NM_005517 | 0.97 | 1.15 | 0.841273347 |
| HMGIY | NM_002131 | 0.92 | 1.04 | 0.886466628 |
| HMGIY | NM_002131 | 0.93 | 1.13 | 0.824826257 |
| HNF-1A | M57732 | 1.03 | 1.19 | 0.868596298 |
| HNF-1A | M57732 | 1.07 | 1.25 | 0.861349263 |
| HNF-1B | X71346 | 2.40 | 2.21 | 1.087117438 |
| HNF-1B | X71346 | 2.25 | 2.18 | 1.030922798 |
| HNF-3gamma | L12141 | 1.46 | 1.53 | 0.956635501 |
| HNF-3gamma | L12141 | 1.40 | 1.54 | 0.90844598 |
| HNF-4alpha3 | U72967 | 2.92 | 3.06 | 0.953909282 |
| HNF-4alpha3 | U72967 | 2.76 | 3.16 | 0.871764387 |
| HNF-6alpha | AF035580 | 1.20 | 1.00 | 1.202677165 |
| HNF-6alpha | AF035580 | 1.02 | 1.07 | 0.954515537 |
| HNF3A | NM_004496 | 1.35 | 1.39 | 0.968770391 |
| HNF3A | NM_004496 | 1.30 | 1.39 | 0.934312714 |
| HOX | L11239 | 1.29 | 1.55 | 0.831459424 |
| HOX | L11239 | 1.22 | 1.56 | 0.784287548 |
| HOX11 | s38742 | 0.82 | 0.97 | 0.846268344 |
| HOX11 | s38742 | 0.89 | 1.06 | 0.840219605 |
| HOX11L2 | AJ223798 | 5.90 | 5.44 | 1.08601856 |
| HOX11L2 | AJ223798 | 5.29 | 5.47 | 0.967027069 |
| HOXA-9 | U81511 | 2.28 | 2.06 | 1.107860869 |
| HOXA-9 | U81511 | 2.06 | 2.02 | 1.019494694 |
| HOXA1 | S79910 | 1.47 | 1.44 | 1.023612925 |
| HOXA1 | S79910 | 1.22 | 1.31 | 0.930731462 |
| HOXA11 | AF071164 | 1.23 | 1.36 | 0.902672948 |
| HOXA11 | AF071164 | 1.28 | 1.48 | 0.86247018 |
| HOXA13 | NM_000522 | 7.13 | 5.19 | 1.375914112 |
| HOXA13 | NM_000522 | 3.90 | 4.45 | 0.876388041 |
| HOXA4 | U56105 | 1.20 | 1.41 | 0.854164123 |
| HOXA4 | U56105 | 1.19 | 1.46 | 0.814779811 |
| HOXA7 | NM_006896 | 1.14 | 1.20 | 0.952764133 |
| HOXA7 | NM_006896 | 1.09 | 1.21 | 0.899003953 |
| HOXB1 | X16666 | 1.59 | 1.81 | 0.877682176 |
| HOXB1 | X16666 | 1.62 | 2.00 | 0.80887332 |
| HOXB2 | X78978 | 1.84 | 1.60 | 1.145917 |
| HOXB2 | X78978 | 1.64 | 1.72 | 0.957991608 |
| HOXB2 | X16665 | 1.39 | 1.54 | 0.905368978 |
| HOXB2 | X16665 | 1.42 | 1.59 | 0.895429132 |
| HOXB3 | X16667 | 1.92 | 1.73 | 1.107588304 |
| HOXB3 | X16667 | 1.87 | 1.84 | 1.015740013 |
| HOXB4 | AF005652 | 1.16 | 1.27 | 0.911652213 |
| HOXB4 | AF005652 | 1.09 | 1.24 | 0.880915725 |
| HOXB5 | M92299 | 1.18 | 1.38 | 0.854344138 |
| HOXB5 | M92299 | 1.20 | 1.49 | 0.803737757 |
| HOXB7 | M16937 | 0.95 | 1.22 | 0.778800068 |
| HOXB7 | M16937 | 0.97 | 1.24 | 0.778387715 |
| HOXC10 | AF255675 | 1.16 | 1.28 | 0.905053085 |
| HOXC10 | X99685 | 1.12 | 1.27 | 0.881270065 |
| HOXC10 | AF255675 | 1.13 | 1.31 | 0.858450467 |
| HOXC10 | X99685 | 1.10 | 1.33 | 0.82796661 |
| HOXC6 | M16938 | 1.26 | 1.49 | 0.844466889 |
| HOXC6 | M16938 | 1.16 | 1.46 | 0.800039127 |
| HOXC8 | X99681 | 1.12 | 1.30 | 0.860768554 |
| HOXC8 | X99681 | 0.97 | 1.24 | 0.783209726 |
| HOXD3 | NM_006898 | 1.51 | 1.62 | 0.92856985 |
| HOXD3 | NM_006898 | 1.47 | 1.61 | 0.918716026 |
| HOXD4 | X04706 | 1.24 | 1.40 | 0.886519344 |
| HOXD4 | X67079 | 1.56 | 1.78 | 0.877418885 |
| HOXD4 | X67079 | 1.54 | 1.86 | 0.826005297 |
| HOXD4 | X04706 | 1.22 | 1.52 | 0.804048475 |
| HPX42B | NM_014468 | 1.02 | 1.04 | 0.980963071 |
| HPX42B | NM_014468 | 0.91 | 1.00 | 0.913143774 |
| hRev | X72631 | 1.25 | 1.35 | 0.929674185 |
| hRev | X72631 | 1.28 | 1.42 | 0.902255362 |
| HS747E2A | NM_015370 | 1.07 | 1.12 | 0.959032318 |
| HS747E2A | NM_015370 | 1.02 | 1.17 | 0.873166624 |
| HSA275986 | NM_018403 | 1.80 | 1.66 | 1.081002809 |
| HSA275986 | NM_018403 | 1.61 | 1.81 | 0.888060724 |
| HSBP1 | AF068754 | 2.24 | 2.62 | 0.853507954 |
| HSBP1 | AF068754 | 2.27 | 2.83 | 0.801085361 |
| HSET | D14678 | 0.47 | 0.56 | 0.84140568 |
| HSET | D14678 | 0.46 | 0.59 | 0.779570541 |
| HSF2BP | NM_007031 | 2.36 | 2.61 | 0.904409562 |
| HSF2BP | NM_007031 | 2.24 | 2.57 | 0.86866997 |
| HSGT1 | NM_007265 | 1.14 | 1.17 | 0.973056944 |
| HSGT1 | NM_007265 | 1.12 | 1.27 | 0.878498082 |
| hSIM2 | D85922 | 2.71 | 2.85 | 0.952407887 |
| hSIM2 | D85922 | 2.65 | 2.91 | 0.910509622 |
| Hsp90 | X07270 | 0.92 | 1.11 | 0.82588322 |
| Hsp90 | X15183 | 2.01 | 2.48 | 0.812100632 |
| hTFIIS.h | AJ223473 | 0.99 | 1.13 | 0.878742961 |
| hTFIIS.h | AJ223473 | 0.98 | 1.14 | 0.856298131 |
| HUNKI | Y12059 | 1.59 | 1.62 | 0.976707993 |
| HUNKI | Y12059 | 1.33 | 1.50 | 0.884627755 |
| HZF2 | X78925 | 1.12 | 1.19 | 0.948487222 |
| HZF2 | X78925 | 1.08 | 1.19 | 0.908973223 |
| HZF3 | X78926 | 1.28 | 1.39 | 0.920945575 |
| HZF3 | X78926 | 1.10 | 1.31 | 0.838730175 |
| HZF8 | X78931 | 1.56 | 1.52 | 1.022134201 |
| HZF8 | X78931 | 1.40 | 1.56 | 0.896953681 |
| HZF9 | X78932 | 1.14 | 1.24 | 0.918602524 |
| HZF9 | X78932 | 1.11 | 1.30 | 0.857126824 |
| Id1 | NM_002165 | 1.24 | 1.23 | 1.00902126 |
| Id1 | NM_002165 | 1.13 | 1.41 | 0.80522294 |
| Id3 | A17548 | 1.38 | 1.31 | 1.055781754 |
| Id3 | X69111 | 1.27 | 1.28 | 0.990641606 |
| Id4 | Y07958 | 1.15 | 1.26 | 0.913664616 |
| Id4 | Y07958 | 1.09 | 1.32 | 0.830113526 |
| InsAF | s73205 | 1.84 | 2.05 | 0.898920183 |
| InsAF | s73205 | 1.85 | 2.13 | 0.871765981 |
| intergenic region | U15407 | 2.30 | 2.60 | 0.88468389 |
| HOXB7-HOXB6 | | | | |
| intergenic region | U15407 | 2.04 | 2.59 | 0.785453268 |
| HOXB7-HOXB6 | | | | |
| IQGAP2 | NM_006633 | 1.12 | 1.12 | 0.998484582 |
| IQGAP2 | NM_006633 | 0.94 | 1.12 | 0.840859025 |
| IRF-1 | X14454 | 2.41 | 2.57 | 0.938218115 |
| IRF-1 | X14454 | 2.39 | 2.58 | 0.925343204 |
| IRF2 | NM_002199 | 3.34 | 2.85 | 1.173965009 |
| IRF2 | NM_002199 | 2.94 | 2.56 | 1.14907375 |
| IRF4 | U52682 | 1.32 | 1.28 | 1.029933166 |
| IRF4 | U52682 | 1.37 | 1.43 | 0.959410817 |
| IRF5 | NM_002200 | 1.37 | 1.51 | 0.904052621 |
| IRF5 | NM_002200 | 1.36 | 1.59 | 0.858607001 |
| IRF6 | NM_006147 | 1.29 | 1.58 | 0.813425333 |
| IRF6 | NM_006147 | 1.18 | 1.50 | 0.789190299 |
| IRF7 | U53830 | 1.84 | 1.44 | 1.27973546 |
| IRF7 | NM_004029 | 1.32 | 1.21 | 1.084000454 |
| Irx-4 | NM_016358 | 1.19 | 1.15 | 1.029933166 |
| Irx-4 | NM_016358 | 1.17 | 1.22 | 0.956334448 |
| IsGF-3gamma | M87503 | 1.42 | 1.55 | 0.915149715 |
| IsGF-3gamma | M87503 | 1.39 | 1.56 | 0.887975373 |
| Jun-D | X56681 | 2.38 | 2.25 | 1.056280294 |
| Jun-D | X56681 | 2.04 | 2.18 | 0.933938896 |
| JunB | X51345 | 1.02 | 1.14 | 0.892190868 |
| JunB | X51345 | 0.98 | 1.14 | 0.855272625 |
| K-ALPHA-1 | NM_006082 | 0.83 | 0.96 | 0.86884485 |
| K-ALPHA-1 | NM_006082 | 0.83 | 0.97 | 0.859281424 |
| KF1 | NM_005667 | 0.93 | 1.05 | 0.890983333 |
| KF1 | NM_005667 | 0.91 | 1.06 | 0.864474263 |
| KIAA0048 | D28588 | 1.17 | 1.24 | 0.943988673 |
| KIAA0048 | D28588 | 1.19 | 1.30 | 0.918453567 |
| KIAA0065 | D31763 | 2.61 | 2.47 | 1.058679492 |
| KIAA0065 | D31763 | 2.53 | 2.52 | 1.005681703 |
| KIAA0071 | NM_015156 | 2.49 | 2.21 | 1.124047572 |
| KIAA0071 | NM_015156 | 2.30 | 2.27 | 1.015956269 |
| KIAA0130 | NM_014815 | 1.35 | 1.36 | 0.9886418 |
| KIAA0130 | NM_014815 | 1.17 | 1.34 | 0.869733568 |
| KIAA0161 | D79983 | 1.43 | 1.66 | 0.85937708 |
| K1AA0161 | D79983 | 1.42 | 1.69 | 0.837823111 |
| KIAA0211 | D86966 | 1.41 | 1.67 | 0.846204986 |
| KIAA0211 | D86966 | 1.37 | 1.73 | 0.79442123 |
| KIAA0222 | D86975 | 2.22 | 2.40 | 0.925360475 |
| KIAA0222 | D86975 | 2.02 | 2.43 | 0.82835128 |
| KIAA0244 | NM_015153 | 1.54 | 1.39 | 1.1095751 |
| KIAA0244 | NM_015153 | 1.45 | 1.36 | 1.067040755 |
| KIAA0314 | AB002312 | 2.38 | 2.57 | 0.927343337 |
| KIAA0314 | AB002312 | 2.33 | 2.65 | 0.876030662 |
| KIAA0333 | AB002331 | 1.05 | 1.22 | 0.861487483 |
| KIAA0333 | AB002331 | 1.07 | 1.25 | 0.854015656 |
| KIAA0352 | NM_014830 | 2.88 | 3.18 | 0.9057295 |
| KIAA0352 | NM_014830 | 2.37 | 2.80 | 0.8492877 |
| KIAA0395 | AB007855 | 1.56 | 1.77 | 0.879373168 |
| KIAA0395 | AB007855 | 1.42 | 1.77 | 0.801179995 |
| KIAA0426 | NM_014724 | 1.17 | 1.17 | 0.995781911 |
| KIAA0426 | NM_014724 | 1.06 | 1.23 | 0.866553199 |
| KIAA0478 | AB007947 | 2.27 | 2.38 | 0.954072874 |
| KIAA0478 | AB007947 | 2.25 | 2.62 | 0.857934327 |
| KIAA0569 | NM_014795 | 1.66 | 1.65 | 1.011174941 |
| KIAA0569 | NM_014795 | 1.39 | 1.76 | 0.78836631 |
| KIAA0595 | AB011167 | 1.90 | 1.85 | 1.026787356 |
| KIAA0595 | AB011167 | 1.71 | 2.19 | 0.782500333 |
| KIAA0600 | AB011172 | 1.90 | 1.34 | 1.413460448 |
| KIAA0600 | AB011172 | 2.18 | 1.58 | 1.381300612 |
| KIAA0929 | AB023146 | 1.54 | 1.62 | 0.949493335 |
| KIAA0929 | AB023146 | 1.55 | 1.63 | 0.949132006 |
| KIAA1015 | AB023232 | 2.58 | 2.62 | 0.982939758 |
| KIAA1015 | AB023232 | 2.17 | 2.68 | 0.811021231 |
| KIAA1259 | AB033085 | 0.85 | 1.04 | 0.817749165 |
| KIAA1259 | AB033085 | 0.91 | 1.18 | 0.771007682 |
| KIAA1442 | AB037863 | 2.14 | 2.34 | 0.914709549 |
| KIAA1442 | AB037863 | 2.15 | 2.39 | 0.898110711 |
| KIAA1528 | AB040961 | 6.42 | 6.40 | 1.003589265 |
| KIAA1528 | AB040961 | 6.67 | 6.98 | 0.955545485 |
| KIAA1741 | AW081989 | 1.58 | 1.79 | 0.882769399 |
| KIAA1741 | AW081989 | 1.68 | 1.99 | 0.846731464 |
| KID | D38751 | 1.54 | 1.48 | 1.042612741 |
| KID | D38751 | 1.45 | 1.52 | 0.95955196 |
| KLF13 | NM_015995 | 1.04 | 1.28 | 0.816419879 |
| KLF13 | NM_015995 | 0.91 | 1.14 | 0.796485569 |
| KNSL4 | AB017335 | 1.22 | 1.41 | 0.866676983 |
| KNSL4 | AB017335 | 1.19 | 1.45 | 0.818446687 |
| Kox1 | X52332 | 1.02 | 1.16 | 0.880125266 |
| Kox1 | X52332 | 0.98 | 1.24 | 0.789133958 |
| Kox23 | X52354 | 0.91 | 1.08 | 0.842330108 |
| Kox23 | X52354 | 0.90 | 1.08 | 0.832659332 |
| Kox26 | X52357 | 1.00 | 1.19 | 0.83622347 |
| Kox26 | X52357 | 0.99 | 1.26 | 0.785398373 |
| Kox29 | X52360 | 0.96 | 1.07 | 0.90087877 |
| Kox29 | X52360 | 0.98 | 1.09 | 0.897521031 |
| Kox30 | X52361 | 1.58 | 1.72 | 0.918425379 |
| Kox30 | X52361 | 1.38 | 1.53 | 0.902401118 |
| KRAB | M67508 | 1.56 | 1.63 | 0.955633904 |
| KRAB | M67508 | 1.47 | 1.60 | 0.922478172 |
| Kruppel-type ZNF | AJ245587 | 2.04 | 2.40 | 0.851750841 |
| Kruppel-type ZNF | AJ245587 | 1.79 | 2.14 | 0.836843037 |
| KUP | X16576 | 0.96 | 1.15 | 0.839112982 |
| KUP | X16576 | 0.92 | 1.13 | 0.816714046 |
| L-Myc-1(long form) | X07262 | 1.05 | 1.20 | 0.876584744 |
| L-Myc-1(long form) | X07262 | 1.01 | 1.23 | 0.826416004 |
| LAF4 | NM_002285 | 0.67 | 0.83 | 0.815483937 |
| LAF4 | NM_002285 | 0.65 | 0.84 | 0.784014372 |
| LBR | NM_002296 | 1.25 | 1.30 | 0.966371608 |
| LBR | NM_002296 | 1.23 | 1.38 | 0.891519857 |
| LD5-1 | U88080 | 1.15 | 1.38 | 0.82971758 |
| LD5-1 | U88080 | 1.11 | 1.41 | 0.790825308 |
| LDOC1 | NM_012317 | 1.33 | 1.41 | 0.946393907 |
| LDOC1 | NM_012317 | 1.28 | 1.42 | 0.897325005 |
| LEF-1 | AF203908 | 1.27 | 1.37 | 0.928294795 |
| LEF-1 | AF203908 | 1.16 | 1.44 | 0.810978586 |
| lens epithelium-derived | AF063020 | 1.24 | 1.42 | 0.870186854 |
| GF | | | | |
| lens epithelium-derived | AF063020 | 1.13 | 1.39 | 0.81400662 |
| GF | | | | |
| leucine zipper | AF056184 | 2.24 | 2.72 | 0.824441293 |
| leucine zipper | AF056184 | 2.47 | 3.10 | 0.796652442 |
| leucine zipper kinase | AF251441 | 2.80 | 3.31 | 0.846204986 |
| AZK | | | | |
| leucine zipper kinase | AF251441 | 2.71 | 3.35 | 0.808081689 |
| AZK | | | | |
| LHX2 | NM_004789 | 1.42 | 1.48 | 0.953866869 |
| LHX2 | NM_004789 | 1.33 | 1.52 | 0.87550605 |
| LHX6 | NM_014368 | 1.31 | 1.42 | 0.921284172 |
| LHX6 | NM_014368 | 1.28 | 1.42 | 0.905610681 |
| LIM | AF061258 | 1.13 | 1.44 | 0.78655152 |
| LIM | AF061258 | 1.09 | 1.41 | 0.773071315 |
| LIM domain only 1 | M26682 | 1.39 | 1.44 | 0.966564434 |
| (rhombotin 1) | | | | |
| LIM domain only 1 | M26682 | 1.32 | 1.49 | 0.883805842 |
| (rhombotin 1) | | | | |
| LIM protein MLP | U49837 | 0.96 | 1.03 | 0.937706079 |
| LIM protein MLP | U49837 | 0.95 | 1.14 | 0.82868354 |
| LIM1 | U14755 | 1.17 | 1.23 | 0.952256263 |
| LIM1 | U14755 | 1.01 | 1.27 | 0.798369687 |
| LIMK | D26309 | 2.92 | 3.03 | 0.964180024 |
| LIMK-2 | D45906 | 1.60 | 1.66 | 0.965944664 |
| LIMK-2 | D45906 | 1.63 | 1.73 | 0.945399728 |
| LMO4 | U24576 | 0.85 | 0.84 | 1.007125772 |
| LMO4 | U24576 | 0.85 | 0.88 | 0.960803963 |
| LOC51043 | NM_015872 | 0.86 | 0.91 | 0.949928525 |
| LOC51043 | NM_015872 | 0.96 | 1.02 | 0.943797482 |
| LOC51131 | NM_016119 | 1.08 | 1.04 | 1.041898041 |
| LOC51131 | NM_016119 | 1.01 | 1.03 | 0.974830053 |
| LOC51193 | NM_016331 | 1.18 | 1.40 | 0.846337164 |
| LOC51193 | NM_016331 | 1.26 | 1.54 | 0.817829826 |
| LOC51591 | NM_015905 | 5.44 | 4.01 | 1.354983586 |
| LOC51591 | NM_015905 | 5.77 | 4.26 | 1.353823958 |
| LOC51717 | NM_016285 | 1.43 | 1.55 | 0.919576048 |
| LOC51717 | NM_016285 | 1.32 | 1.54 | 0.856030646 |
| LOC55862 | NM_018479 | 3.18 | 3.29 | 0.96566812 |
| LOC55862 | NM_018479 | 2.90 | 3.29 | 0.882904561 |
| LOC56899 | AF164792 | 1.35 | 1.46 | 0.923685155 |
| LOC56899 | AF164792 | 1.24 | 1.47 | 0.843151755 |
| LyF-1 | U40462 | 1.17 | 1.33 | 0.881798663 |
| LyF-1 | U40462 | 1.04 | 1.28 | 0.809448886 |
| LZLP | NM_013344 | 1.63 | 1.78 | 0.914937922 |
| LZLP | NM_013344 | 1.50 | 1.67 | 0.897409878 |
| MADH4 | NM_005359 | 1.48 | 1.23 | 1.202041185 |
| MADH4 | NM_005359 | 1.27 | 1.13 | 1.118588098 |
| MADH5 | NM_005903 | 1.19 | 1.37 | 0.867598314 |
| MADH5 | NM_005903 | 1.20 | 1.38 | 0.864484722 |
| MAF | NM_005360 | 0.82 | 0.83 | 0.983383327 |
| MAF | NM_005360 | 0.74 | 0.92 | 0.79706277 |
| MAFG | NM_002359 | 1.33 | 1.60 | 0.833961234 |
| MAFG | NM_002359 | 1.37 | 1.65 | 0.833526497 |
| MAP4 | NM_002375 | 3.80 | 4.62 | 0.824293011 |
| MAP4 | NM_002375 | 3.69 | 4.71 | 0.78417244 |
| MAPK8 | NM_002750 | 0.88 | 1.00 | 0.88152506 |
| MAPK8 | NM_002750 | 0.88 | 1.02 | 0.860049569 |
| MAZ | M94046 | 1.21 | 1.47 | 0.819442731 |
| MAZ | M94046 | 1.19 | 1.48 | 0.804052549 |
| MB67 | Z30425 | 1.08 | 1.02 | 1.060408157 |
| MB67 | Z30425 | 0.99 | 1.08 | 0.915952791 |
| MCG4 | NM_006782 | 1.15 | 1.31 | 0.87362439 |
| MCG4 | NM_006782 | 1.15 | 1.34 | 0.857557298 |
| MEF2A | U49020 | 1.18 | 1.29 | 0.917750293 |
| MEF2A | U49020 | 1.08 | 1.27 | 0.851735738 |
| MEF2B | NM_005919 | 1.02 | 1.07 | 0.950137026 |
| MEF2B | NM_005919 | 0.95 | 1.04 | 0.910069513 |
| MEF2D | NM_005920 | 1.39 | 1.33 | 1.043108425 |
| MEF2D | NM_005920 | 1.20 | 1.44 | 0.837034123 |
| metallopanstimulin | U85979 | 1.98 | 2.01 | 0.985678226 |
| metallopanstimulin | U85979 | 1.94 | 2.20 | 0.882570172 |
| MHox (K-2) | M95929 | 1.07 | 1.17 | 0.914292266 |
| MHox (K-2) | M95929 | 0.95 | 1.17 | 0.810474232 |
| Mi | Z29678 | 1.71 | 1.66 | 1.030471845 |
| Mi | Z29678 | 1.76 | 1.79 | 0.986205176 |
| MITF | AF034755 | 1.23 | 1.24 | 0.99130983 |
| MITF | AF034755 | 1.32 | 1.50 | 0.883057308 |
| Miz-1 | Y09723 | 1.01 | 1.15 | 0.876000186 |
| Miz-1 | Y09723 | 0.93 | 1.14 | 0.814161592 |
| MLH3 | NM_005784 | 0.54 | 0.63 | 0.855999025 |
| MLH3 | NM_005784 | 0.62 | 0.78 | 0.801334083 |
| MLX | AF203978 | 1.41 | 1.49 | 0.949042398 |
| MLX | AF203978 | 1.36 | 1.48 | 0.923306997 |
| Mog | U64564 | 1.32 | 1.37 | 0.960484338 |
| Mog | U64564 | 1.27 | 1.39 | 0.915925265 |
| MRG1 | AF109161 | 3.76 | 4.37 | 0.860312626 |
| MRG1 | AF109161 | 3.73 | 4.50 | 0.827783082 |
| MTERF | NM_006980 | 1.51 | 1.80 | 0.838119573 |
| MTERF | NM_006980 | 1.35 | 1.70 | 0.789625228 |
| MTF-1 | AJ251881 | 2.11 | 2.39 | 0.881959401 |
| MTF-1 | AJ251881 | 1.95 | 2.39 | 0.815353763 |
| mtTF1 | X64269 | 1.47 | 1.59 | 0.925536704 |
| mtTF1 | X64269 | 1.44 | 1.57 | 0.914838473 |
| MXI1 | NM_005962 | 1.16 | 1.29 | 0.898657286 |
| MXI1 | NM_005962 | 1.16 | 1.36 | 0.857867078 |
| MYBBP1A | AF147709 | 2.29 | 1.77 | 1.292847997 |
| MYBBP1A | AF147709 | 1.85 | 1.75 | 1.054649057 |
| MYCBP | NM_012333 | 3.73 | 3.58 | 1.040887845 |
| MYCBP | NM_012333 | 3.47 | 3.48 | 0.997884909 |
| MYCL2 | NM_005377 | 2.12 | 2.03 | 1.044677307 |
| MYCL2 | NM_005377 | 2.04 | 2.00 | 1.018897998 |
| MYCLK1 | M64786 | 1.43 | 1.73 | 0.828883125 |
| MYCLK1 | M64786 | 1.49 | 1.80 | 0.826354974 |
| MYT2 | NM_003871 | 4.01 | 4.17 | 0.962205771 |
| MYT2 | NM_003871 | 4.04 | 4.42 | 0.915182881 |
| N-CoR | AF044209 | 1.33 | 1.29 | 1.027153581 |
| N-CoR | AF044209 | 1.25 | 1.29 | 0.969389141 |
| N-Oct-3 | Z11933 | 3.50 | 3.17 | 1.103689021 |
| N-Oct-3 | Z11933 | 2.91 | 3.05 | 0.955346496 |
| N143 | AJ002572 | 3.89 | 3.16 | 1.232431216 |
| N143 | AJ002572 | 2.82 | 3.41 | 0.828068155 |
| NACA | NM_005594 | 1.34 | 1.26 | 1.061449635 |
| NACA | NM_005594 | 1.22 | 1.36 | 0.899257451 |
| NAGA | NM_000262 | 2.23 | 2.55 | 0.873072079 |
| NAGA | NM_000262 | 2.02 | 2.54 | 0.795967326 |
| NCOA1 | NM_003743 | 1.34 | 1.43 | 0.939022342 |
| NCOA1 | NM_003743 | 1.36 | 1.45 | 0.932646647 |
| NCOA3 | NM_006534 | 2.14 | 2.15 | 0.995002762 |
| NCOA3 | NM_006534 | 1.97 | 2.05 | 0.959254041 |
| NCYM | NM_006316 | 1.18 | 1.11 | 1.067219564 |
| NCYM | NM_006316 | 1.07 | 1.16 | 0.917384574 |
| NDUFA6 | NM_002490 | 0.80 | 0.82 | 0.969899497 |
| NDUFA6 | NM_002490 | 0.71 | 0.92 | 0.772339515 |
| Negative control | Negative control | 1.29 | 1.11 | 1.161392449 |
| Negative control | Negative control | 5.43 | 5.29 | 1.027043989 |
| NEUROD2 | U58681 | 1.14 | 1.28 | 0.889551897 |
| NEUROD2 | U58681 | 1.02 | 1.28 | 0.795592113 |
| NEUROG1 | U63842 | 1.39 | 1.71 | 0.812574039 |
| NEUROG1 | U63842 | 1.29 | 1.63 | 0.795487149 |
| NF-1X | U07811 | 0.99 | 0.82 | 1.215806558 |
| NF-1X | U07811 | 0.64 | 0.82 | 0.782275487 |
| NFAT1 | U43341 | 2.28 | 2.65 | 0.861852199 |
| NFAT1 | U43341 | 2.30 | 2.80 | 0.819721245 |
| NFATC1 | NM_006162 | 1.21 | 1.27 | 0.956885723 |
| NFATC1 | NM_006162 | 1.20 | 1.33 | 0.906442678 |
| NFATX | U14510 | 1.09 | 1.35 | 0.8066644 |
| NFATX | U14510 | 0.99 | 1.24 | 0.798995238 |
| NFIL3 | NM_005384 | 3.33 | 3.43 | 0.969982487 |
| NFIL3 | NM_005384 | 3.22 | 3.36 | 0.957589194 |
| NFKB1 | M58603 | 2.44 | 2.68 | 0.910234175 |
| NFKB1 | M55643 | 1.23 | 1.37 | 0.894069494 |
| NFKB2 | U09609 | 1.09 | 1.21 | 0.899003953 |
| NFKB2 | U09609 | 1.04 | 1.28 | 0.815426014 |
| NFKBIB | NM_002503 | 0.66 | 0.74 | 0.891632657 |
| NFKBIB | NM_002503 | 0.61 | 0.73 | 0.835589511 |
| NFKBIE | NM_004556 | 1.34 | 1.34 | 0.995844935 |
| NFKBIE | NM_004556 | 1.30 | 1.37 | 0.951450999 |
| NFkBp105 | M55643 | 0.82 | 0.80 | 1.028950235 |
| NFkBp105 | M55643 | 0.78 | 0.88 | 0.882630106 |
| ngn3 | AJ133776 | 1.04 | 1.14 | 0.911675496 |
| ngn3 | AJ133776 | 1.01 | 1.15 | 0.877939013 |
| NME2 | NM_002512 | 1.08 | 1.28 | 0.849242645 |
| NME2 | NM_002512 | 0.99 | 1.21 | 0.819907039 |
| Nmi | U32849 | 1.50 | 1.65 | 0.908824603 |
| Nmi | U32849 | 1.46 | 1.67 | 0.874987469 |
| NOD1 | AF149774 | 0.88 | 0.97 | 0.913819737 |
| NOD1 | AF149774 | 0.80 | 0.97 | 0.829329103 |
| NOT3 | NM_014516 | 1.10 | 1.05 | 1.043010425 |
| NOT3 | NM_014516 | 1.01 | 1.32 | 0.770565768 |
| NP220 | D83032 | 1.59 | 1.80 | 0.886060234 |
| NP220 | D83032 | 1.50 | 1.74 | 0.861556367 |
| NPAS1 | NM_002517 | 2.55 | 3.06 | 0.832115639 |
| NPAS1 | NM_002517 | 2.53 | 3.18 | 0.795687598 |
| NR0B1 | NM_000475 | 0.89 | 0.90 | 0.985394227 |
| NR0B1 | NM_000475 | 0.84 | 1.02 | 0.822398708 |
| NR2F6 | NM_005234 | 1.11 | 1.26 | 0.878406569 |
| NR2F6 | NM_005234 | 1.03 | 1.23 | 0.843593242 |
| NR3C1 | NM_000176 | 1.45 | 1.63 | 0.884627755 |
| NR3C1 | NM_000176 | 1.37 | 1.57 | 0.872392013 |
| NR4A2 | NM_006186 | 5.15 | 5.52 | 0.933470433 |
| NR4A2 | NM_006186 | 4.88 | 5.68 | 0.859257687 |
| NR5A1 | NM_004959 | 1.55 | 1.88 | 0.827098402 |
| NR5A1 | NM_004959 | 1.56 | 1.94 | 0.806250074 |
| NRL | M81840 | 1.12 | 1.36 | 0.824507422 |
| NRL | M81840 | 1.08 | 1.34 | 0.802675923 |
| NRsF form 2 | U13879 | 1.51 | 1.60 | 0.940689035 |
| NRsF form 2 | U13879 | 1.29 | 1.56 | 0.826356612 |
| NSEP1 | NM_004559 | 4.08 | 4.54 | 0.898882683 |
| NSEP1 | NM_004559 | 4.09 | 4.85 | 0.843016697 |
| nuclear factor 1 B-type | U07810 | 1.67 | 1.72 | 0.970651102 |
| nuclear factor 1 B-type | U07810 | 1.50 | 1.56 | 0.957498055 |
| nuclear factor I-B2 | U85193 | 5.86 | 6.80 | 0.86191166 |
| nuclear factor I-B2 | U85193 | 6.04 | 7.09 | 0.85215085 |
| nuclear factor IV | X57500 | 1.44 | 1.28 | 1.118113871 |
| nuclear factor IV | X57500 | 1.35 | 1.53 | 0.882482444 |
| OAZ | AF221712 | 0.95 | 0.98 | 0.974744849 |
| OAZ | AF221712 | 0.82 | 0.94 | 0.867200363 |
| Oct-1B = POU | S66902 | 1.11 | 1.23 | 0.901795827 |
| homeodomain | | | | |
| Oct-1B = POU | S66902 | 1.07 | 1.22 | 0.879755632 |
| homeodomain | | | | |
| Oct-4A | Z11900 | 1.44 | 1.75 | 0.825538314 |
| Oct-4A | Z11900 | 1.43 | 1.85 | 0.773109431 |
| OGL12 | AF023203 | 2.31 | 2.68 | 0.864811744 |
| OGL12 | AF023203 | 2.45 | 2.92 | 0.837215348 |
| OSMRB | U60805 | 0.90 | 1.09 | 0.825581 |
| OSMRB | U60805 | 0.78 | 0.97 | 0.805457565 |
| OTF3C | Z11901 | 6.19 | 4.42 | 1.40097838 |
| OTF3C | Z11901 | 5.89 | 4.42 | 1.332945525 |
| OTX1 | AB037501 | 1.87 | 1.84 | 1.018457389 |
| OTX1 | AB037501 | 1.75 | 1.86 | 0.938379 |
| OVOL1 | NM_004561 | 1.34 | 1.20 | 1.12018921 |
| OVOL1 | NM_004561 | 1.05 | 1.21 | 0.869990813 |
| p130 | s67171 | 2.11 | 1.71 | 1.231079229 |
| p130 | s67171 | 1.56 | 1.60 | 0.975948711 |
| p243 | AJ242977 | 1.27 | 1.43 | 0.884227005 |
| p243 | AJ242977 | 1.37 | 1.69 | 0.812266478 |
| P38IP | NM_017569 | 0.99 | 1.09 | 0.90720651 |
| P38IP | NM_017569 | 0.94 | 1.10 | 0.856087428 |
| p53 | K03199 | 1.39 | 1.68 | 0.831158406 |
| p53 | K03199 | 1.38 | 1.67 | 0.828166161 |
| p621 | AJ242978 | 1.19 | 1.20 | 0.990516633 |
| p621 | AJ242978 | 1.06 | 1.18 | 0.898178687 |
| PACE4 | NM_002570 | 1.29 | 1.48 | 0.867988727 |
| PACE4 | NM_002570 | 1.26 | 1.48 | 0.852795758 |
| PAX1 | NM_006192 | 1.20 | 1.32 | 0.903818349 |
| PAX1 | NM_006192 | 1.06 | 1.32 | 0.807894213 |
| PAX2 | U45255 | 1.46 | 1.62 | 0.901165711 |
| PAX2 | U45255 | 1.40 | 1.63 | 0.858751434 |
| PAX3 | NM_000438 | 1.21 | 1.36 | 0.886358667 |
| PAX3 | NM_000438 | 1.16 | 1.37 | 0.850478749 |
| PAX5 | U56835 | 0.93 | 1.06 | 0.871188843 |
| PAX5 | NM_016734 | 1.71 | 2.02 | 0.846266 |
| PAX6 | U63833 | 1.33 | 1.53 | 0.865756264 |
| PAX6 | U63833 | 1.27 | 1.52 | 0.833925287 |
| PAX8 | S55490 | 1.94 | 2.07 | 0.934433059 |
| PAX8 | S55490 | 1.82 | 2.05 | 0.885815601 |
| PAX9 | NM_006194 | 0.78 | 0.95 | 0.817959194 |
| PAX9 | X92850 | 1.17 | 1.49 | 0.784900153 |
| PBX1 | NM_002585 | 1.46 | 1.26 | 1.160624187 |
| PBX1 | NM_002585 | 1.56 | 1.36 | 1.14486291 |
| PBX2 | NM_002586 | 1.14 | 1.28 | 0.885932245 |
| PBX2 | NM_002586 | 1.14 | 1.34 | 0.848773413 |
| PC4 | NM_006713 | 0.70 | 0.80 | 0.879994421 |
| PC4 | NM_006713 | 0.70 | 0.81 | 0.86301099 |
| PCAF | NM_003884 | 1.07 | 1.34 | 0.798415415 |
| PCAF | NM_003884 | 1.03 | 1.29 | 0.796481403 |
| PDEF | NM_012391 | 1.17 | 1.33 | 0.876561702 |
| PDEF | NM_012391 | 1.38 | 1.70 | 0.811137032 |
| PEA3 | D12765 | 1.21 | 1.54 | 0.784602478 |
| PEA3 | D12765 | 1.21 | 1.56 | 0.775569587 |
| PEPD | J04605 | 0.66 | 0.78 | 0.8570049 |
| PEPD | J04605 | 0.71 | 0.86 | 0.827558815 |
| PGF | NM_002632 | 1.08 | 1.18 | 0.917061973 |
| PGF | NM_002632 | 1.01 | 1.20 | 0.83593402 |
| pGLI3HH | M20674 | 1.21 | 1.31 | 0.921799298 |
| pGLI3HH | M20674 | 1.13 | 1.32 | 0.856812924 |
| PIAS3 | NM_006099 | 3.47 | 4.09 | 0.849170482 |
| PIAS3 | NM_006099 | 3.59 | 4.26 | 0.842175439 |
| PINCH | U09284 | 1.62 | 1.49 | 1.088214315 |
| PINCH | U09284 | 1.44 | 1.44 | 1.001116263 |
| Pit-1 | D10216 | 2.56 | 2.74 | 0.934572932 |
| Pit-1 | D10216 | 2.26 | 2.72 | 0.831778211 |
| PITX1 | NM_002653 | 1.04 | 1.23 | 0.841903944 |
| PITX1 | NM_002653 | 0.95 | 1.22 | 0.780247958 |
| PITX2 | U69961 | 2.17 | 1.90 | 1.142972468 |
| PITX2 | U69961 | 1.90 | 1.73 | 1.099447256 |
| PITX3 | NM_005029 | 1.08 | 1.19 | 0.908904038 |
| PITX3 | NM_005029 | 1.05 | 1.16 | 0.900257494 |
| PKNOX1 | NM_004571 | 2.66 | 2.91 | 0.915727678 |
| PKNOX1 | NM_004571 | 2.43 | 2.80 | 0.867326045 |
| PLCG1 | NM_002660 | 0.88 | 1.09 | 0.801330479 |
| PLCG1 | NM_002660 | 0.87 | 1.12 | 0.777390419 |
| PML | M79462 | 2.93 | 3.22 | 0.90918611 |
| PML | M79462 | 2.83 | 3.18 | 0.891376967 |
| POU6F1 | NM_002702 | 1.18 | 1.38 | 0.853348217 |
| POU6F1 | NM_002702 | 1.11 | 1.39 | 0.803725887 |
| PPAR delta | AF187850 | 1.68 | 2.05 | 0.817974153 |
| PPAR delta | AF187850 | 1.63 | 2.05 | 0.797208472 |
| PPARbeta | L07592 | 1.12 | 1.30 | 0.860735779 |
| PPARbeta | L07592 | 1.09 | 1.30 | 0.841097109 |
| PPARBP | NM_004774 | 2.49 | 2.42 | 1.028888699 |
| PPARBP | NM_004774 | 2.59 | 2.62 | 0.989587372 |
| PPARG | NM_005037 | 1.76 | 1.92 | 0.919451555 |
| PPARG | NM_005037 | 1.54 | 1.87 | 0.82431469 |
| PPARGC1 | NM_013261 | 4.02 | 4.08 | 0.985608174 |
| PPARGC1 | NM_013261 | 3.61 | 3.97 | 0.910308604 |
| PPIH | NM_006347 | 1.11 | 1.36 | 0.810066673 |
| PPIH | NM_006347 | 1.10 | 1.37 | 0.797849135 |
| pRb | X16439 | 1.29 | 1.40 | 0.923240454 |
| pRb | X16439 | 1.21 | 1.44 | 0.839720657 |
| PRDM4 | NM_012406 | 1.09 | 1.14 | 0.952491603 |
| PRDM4 | NM_012406 | 1.02 | 1.09 | 0.935085892 |
| protein Id4 | U28368 | 1.18 | 1.11 | 1.06450375 |
| protein Id4 | U28368 | 1.29 | 1.23 | 1.054319434 |
| protein p38 | AJ242975 | 1.63 | 1.95 | 0.834950074 |
| protein p38 | AJ242975 | 1.44 | 1.85 | 0.781801717 |
| PRX2 | NM_016307 | 4.82 | 3.63 | 1.326456916 |
| PRX2 | NM_016307 | 4.32 | 4.25 | 1.017671923 |
| PSCDBP | NM_004288 | 0.74 | 0.85 | 0.86992699 |
| PSCDBP | NM_004288 | 0.69 | 0.84 | 0.81936229 |
| PSMC1 | NM_002802 | 1.36 | 1.52 | 0.894759062 |
| PSMC1 | NM_002802 | 1.18 | 1.33 | 0.891456342 |
| PTHR1 | NM_000316 | 1.31 | 1.42 | 0.924499528 |
| PTHR1 | NM_000316 | 1.20 | 1.40 | 0.8556512 |
| PXMP3 | NM_000318 | 1.62 | 2.02 | 0.804068402 |
| PXMP3 | NM_000318 | 1.40 | 1.80 | 0.78066981 |
| PXN | NM_002859 | 2.72 | 2.90 | 0.93925013 |
| PXN | NM_002859 | 2.51 | 2.90 | 0.863012935 |
| rab 13 | X75593 | 1.25 | 1.23 | 1.008775651 |
| rab 13 | X75593 | 1.12 | 1.33 | 0.83974611 |
| RAR-alpha1 | X06614 | 1.43 | 1.62 | 0.88166305 |
| RAR-alpha1 | X06614 | 1.30 | 1.60 | 0.814209521 |
| RAR-b | M96016 | 1.57 | 1.95 | 0.801619789 |
| RAR-b | M96016 | 1.57 | 2.00 | 0.782949951 |
| RARA | NM_000964 | 1.42 | 1.65 | 0.862685131 |
| RARA | NM_000964 | 1.40 | 1.65 | 0.847918555 |
| RARG | NM_000966 | 1.42 | 1.61 | 0.882296859 |
| RARG | NM_000966 | 1.41 | 1.60 | 0.882261145 |
| RB1 | NM_000321 | 0.97 | 1.19 | 0.812728745 |
| RB1 | NM_000321 | 0.96 | 1.20 | 0.800478062 |
| RBL1 | NM_002895 | 2.04 | 2.42 | 0.841470759 |
| RBL1 | NM_002895 | 1.92 | 2.35 | 0.817621225 |
| RBP-L | AB026048 | 0.94 | 0.70 | 1.339824561 |
| RBP-L | AB026048 | 0.80 | 0.71 | 1.133824475 |
| RCL | NM_006443 | 1.26 | 1.39 | 0.906711617 |
| RCL | NM_006443 | 1.24 | 1.39 | 0.891529469 |
| RELA | Z22951 | 0.86 | 0.89 | 0.96567493 |
| RELA | Z22951 | 0.80 | 0.85 | 0.94852389 |
| repressor protein | D30612 | 1.37 | 1.53 | 0.890929984 |
| repressor protein | D30612 | 1.32 | 1.52 | 0.87316143 |
| REQ | NM_006268 | 1.43 | 1.67 | 0.860238236 |
| REQ | NM_006268 | 1.46 | 1.72 | 0.847602428 |
| retinoid X receptor | U66306 | 2.17 | 2.44 | 0.889382828 |
| alpha | | | | |
| retinoid X receptor- | U38480 | 2.07 | 2.33 | 0.889199662 |
| gamma | | | | |
| RFP | NM_006510 | 3.73 | 3.97 | 0.940382915 |
| RFP | NM_006510 | 3.81 | 4.43 | 0.858648769 |
| RFX3 | X76092 | 1.62 | 1.47 | 1.1024024 |
| RFX3 | X76092 | 1.43 | 1.62 | 0.8816817 |
| rhoHP1 | D85815 | 1.42 | 1.33 | 1.069393174 |
| rhoHP1 | D85815 | 1.46 | 1.53 | 0.955107329 |
| RING1 | NM_002931 | 1.42 | 1.59 | 0.896164283 |
| RING1 | NM_002931 | 1.41 | 1.60 | 0.880810591 |
| RLF | NM_012421 | 3.38 | 3.75 | 0.901013305 |
| RLF | NM_012421 | 3.65 | 4.07 | 0.898102049 |
| RNF NY-REN-43 | AF155109 | 1.18 | 1.29 | 0.913146151 |
| RNF NY-REN-43 | AF155109 | 1.16 | 1.43 | 0.811644103 |
| RNF13 | NM_007282 | 1.22 | 1.33 | 0.916119358 |
| RNF13 | NM_007282 | 1.20 | 1.31 | 0.912867135 |
| RNF15 | NM_006355 | 1.29 | 1.45 | 0.893216374 |
| RNF15 | NM_006355 | 1.17 | 1.44 | 0.811128245 |
| RNF4 | NM_002938 | 1.35 | 1.44 | 0.936370857 |
| RNF4 | NM_002938 | 1.32 | 1.45 | 0.907740218 |
| RNF9 | NM_006778 | 1.25 | 1.36 | 0.918123369 |
| RNF9 | NM_006778 | 1.18 | 1.36 | 0.863185084 |
| RNP-specific A | X06347 | 1.31 | 1.39 | 0.94551044 |
| RNP-specific A | X06347 | 1.16 | 1.47 | 0.788038353 |
| RORalpha2 | U04898 | 4.15 | 4.42 | 0.938764319 |
| RORalpha2 | U04898 | 4.03 | 4.29 | 0.938708029 |
| RORbeta | Y08639 | 1.29 | 1.50 | 0.858111801 |
| RORbeta | Y08639 | 1.27 | 1.50 | 0.842642276 |
| RORC | NM_005060 | 1.39 | 1.61 | 0.861315789 |
| RORC | NM_005060 | 1.43 | 1.77 | 0.807520338 |
| RP58 | AJ223321 | 1.34 | 1.40 | 0.953320654 |
| RP58 | AJ223321 | 1.19 | 1.38 | 0.866072097 |
| RPF-1 | U91934 | 1.26 | 1.51 | 0.833565324 |
| RPF-1 | U91934 | 1.23 | 1.50 | 0.822227125 |
| RPL13A | X56932 | 0.87 | 0.88 | 0.991870123 |
| RPL13A | X56932 | 0.77 | 0.87 | 0.883814097 |
| RPL15 | NM_002948 | 1.01 | 1.07 | 0.944600915 |
| RPL15 | NM_002948 | 0.98 | 1.14 | 0.859452181 |
| RPL21 | NM_000982 | 1.60 | 1.53 | 1.04809166 |
| RPL21 | NM_000982 | 1.57 | 1.58 | 0.995425213 |
| RPL23A | NM_000984 | 1.59 | 1.42 | 1.117137899 |
| RPL23A | NM_000984 | 1.46 | 1.38 | 1.059317332 |
| RPL37 | NM_000997 | 1.09 | 1.23 | 0.883744302 |
| RPL37 | NM_000997 | 1.09 | 1.30 | 0.842639916 |
| RPS11 | NM_001015 | 1.55 | 1.32 | 1.171184602 |
| RPS11 | NM_001015 | 1.30 | 1.22 | 1.068789518 |
| RPS19 | NM_001022 | 0.84 | 1.00 | 0.841774594 |
| RPS19 | NM_001022 | 0.86 | 1.05 | 0.819892642 |
| RRN3 | NM_018427 | 1.64 | 1.61 | 1.015843155 |
| RRN3 | NM_018427 | 1.10 | 1.40 | 0.78552954 |
| RUVBL1 | NM_003707 | 1.21 | 1.41 | 0.864080705 |
| RUVBL1 | NM_003707 | 1.15 | 1.43 | 0.805614035 |
| Rx | AF001911 | 1.40 | 1.19 | 1.169408279 |
| Rx | AF001911 | 1.21 | 1.29 | 0.940515001 |
| RXR-alpha | X52773 | 1.14 | 1.20 | 0.95178794 |
| RXR-alpha | X52773 | 1.02 | 1.17 | 0.875212013 |
| RXRB | U00961 | 1.41 | 1.76 | 0.802764083 |
| RXRB | U00961 | 1.32 | 1.64 | 0.802187954 |
| SAFB | NM_002967 | 2.08 | 1.85 | 1.122521568 |
| SAFB | NM_002967 | 1.98 | 1.85 | 1.072239203 |
| SALL1 | NM_002968 | 1.06 | 1.32 | 0.799379966 |
| SALL1 | NM_002968 | 1.09 | 1.37 | 0.794919835 |
| sAP-1a | M85165 | 1.02 | 1.15 | 0.893216374 |
| sAP-1a | M85165 | 0.99 | 1.14 | 0.868660598 |
| SEP3B | AF285109 | 1.36 | 1.52 | 0.895524427 |
| SEP3B | AF285109 | 1.34 | 1.51 | 0.891662954 |
| sF1 | D88155 | 1.24 | 1.23 | 1.006655807 |
| sF1 | D88155 | 0.89 | 1.10 | 0.815406356 |
| SF3A1 | NM_005877 | 0.94 | 1.18 | 0.796947498 |
| SF3A1 | NM_005877 | 0.98 | 1.24 | 0.789354005 |
| SIX1 | X91868 | 1.28 | 1.26 | 1.011940877 |
| SIX1 | X91868 | 1.15 | 1.27 | 0.899649002 |
| SIX6 | AF141651 | 1.31 | 1.51 | 0.866808238 |
| SIX6 | AF141651 | 1.28 | 1.61 | 0.795100662 |
| SKI | NM_003036 | 1.27 | 1.34 | 0.951830965 |
| SKI | NM_003036 | 1.23 | 1.34 | 0.916814067 |
| SKIL | NM_005414 | 1.22 | 1.23 | 0.996060051 |
| SKIL | NM_005414 | 1.18 | 1.23 | 0.965665088 |
| Smad2 | U78726 | 1.54 | 1.70 | 0.902843033 |
| Smad2 | U78726 | 1.52 | 1.74 | 0.876401307 |
| SMARCA3 | NM_003071 | 2.60 | 2.63 | 0.988267744 |
| SMARCA3 | NM_003071 | 2.52 | 2.58 | 0.977550509 |
| SMARCA4 | NM_003072 | 1.20 | 1.41 | 0.850958457 |
| SMARCA4 | NM_003072 | 1.14 | 1.43 | 0.798506046 |
| SMARCC1 | NM_003074 | 1.37 | 1.53 | 0.897049921 |
| SMARCC1 | NM_003074 | 1.31 | 1.51 | 0.867695906 |
| SMARCC2 | NM_003075 | 1.11 | 1.36 | 0.816630385 |
| SMARCC2 | NM_003075 | 1.10 | 1.37 | 0.803295263 |
| SMN1 | U18423 | 2.14 | 2.06 | 1.0410434 |
| SMN1 | U18423 | 1.93 | 2.06 | 0.938075938 |
| SNAP190 | AF032387 | 1.08 | 1.15 | 0.940066948 |
| SNAP190 | AF032387 | 1.19 | 1.34 | 0.88972042 |
| SNAPC3 | NM_003084 | 0.64 | 0.65 | 0.973605848 |
| SNAPC3 | NM_003084 | 0.58 | 0.67 | 0.873988625 |
| snRNP B | X17567 | 0.99 | 0.98 | 1.010085806 |
| snRNP B | X17567 | 0.82 | 0.97 | 0.840177885 |
| SOX10 | AJ001183 | 1.71 | 1.82 | 0.937524016 |
| SOX10 | AJ001183 | 1.59 | 1.78 | 0.894532832 |
| SOX13 | NM_005686 | 1.71 | 1.92 | 0.891384762 |
| SOX13 | NM_005686 | 1.66 | 1.93 | 0.860515571 |
| SOX4 | X70683 | 0.90 | 0.93 | 0.960960313 |
| SOX4 | X70683 | 0.82 | 0.92 | 0.894488762 |
| SOX6 | X65663 | 0.69 | 0.79 | 0.882795517 |
| SOX6 | X65663 | 0.65 | 0.75 | 0.87280897 |
| SOX8 | AF164104 | 1.79 | 2.09 | 0.857375717 |
| SOX8 | AF164104 | 1.65 | 2.13 | 0.774778844 |
| SOX9 | Z46629 | 1.69 | 1.88 | 0.898185589 |
| SOX9 | Z46629 | 1.55 | 1.92 | 0.807916038 |
| SP1 | J03133 | 1.18 | 1.30 | 0.909375062 |
| SP1 | J03133 | 1.16 | 1.30 | 0.887824726 |
| SP3 | X68560 | 1.66 | 1.66 | 1.001079125 |
| SP3 | X68560 | 1.45 | 1.77 | 0.818395433 |
| sRF | J03161 | 1.45 | 1.67 | 0.867315899 |
| sRF | J03161 | 1.43 | 1.69 | 0.84824421 |
| sRY | L10101 | 1.18 | 1.21 | 0.982441028 |
| sRY | L10101 | 1.13 | 1.21 | 0.934783803 |
| STAT2 | M97934 | 1.41 | 1.52 | 0.926980567 |
| STAT2 | M97934 | 1.41 | 1.62 | 0.868724958 |
| STAT5B | NM_012448 | 1.56 | 1.40 | 1.114847778 |
| STAT5B | NM_012448 | 1.40 | 1.71 | 0.821978259 |
| STAT6 | NM_003153 | 1.27 | 1.36 | 0.938049629 |
| STAT6 | NM_003153 | 1.21 | 1.37 | 0.879605458 |
| SZF1 | NM_016089 | 1.21 | 1.50 | 0.802961061 |
| SZF1 | NM_016089 | 1.16 | 1.49 | 0.774800507 |
| T-STAR | NM_006558 | 0.67 | 0.78 | 0.861849244 |
| T-STAR | NM_006558 | 0.68 | 0.82 | 0.831664605 |
| T3R | Y00479 | 1.71 | 1.62 | 1.053389262 |
| T3R | Y00479 | 1.60 | 1.53 | 1.041322337 |
| T3R | X55066 | 1.56 | 1.89 | 0.824459774 |
| TAF(I)63 | L39061 | 1.00 | 1.12 | 0.896350467 |
| TAF(I)63 | L39061 | 1.02 | 1.30 | 0.783696377 |
| TAF(II)30 | U25816 | 1.51 | 1.40 | 1.074026032 |
| TAF(II)30 | U25816 | 1.40 | 1.38 | 1.015134059 |
| TAF(II)32 | U21858 | 0.98 | 1.22 | 0.802461769 |
| TAF(II)32 | U21858 | 0.98 | 1.23 | 0.792812413 |
| TAF(II)70-alpha | L25444 | 0.96 | 1.02 | 0.941312641 |
| TAF(II)70-alpha | L25444 | 0.90 | 1.03 | 0.872787029 |
| TAF2A | NM_004606 | 1.13 | 1.14 | 0.999330892 |
| TAF2A | NM_004606 | 0.93 | 1.11 | 0.838249213 |
| TAF2F | NM_005642 | 1.03 | 1.26 | 0.81530342 |
| TAF2F | NM_005642 | 1.02 | 1.30 | 0.784947723 |
| TAF2I | NM_005643 | 1.50 | 1.43 | 1.045218429 |
| TAF2I | NM_005643 | 1.39 | 1.41 | 0.991353741 |
| TAF2I | AF118094 | 1.11 | 1.26 | 0.881120736 |
| TAF2J | NM_005644 | 1.28 | 1.40 | 0.913232427 |
| TAF2J | NM_005644 | 1.23 | 1.45 | 0.849684168 |
| TAF2K | NM_005645 | 2.39 | 2.40 | 0.997067405 |
| TAF2K | NM_005645 | 2.40 | 2.47 | 0.972697492 |
| TAFII105 | Y09321 | 1.26 | 1.45 | 0.867396208 |
| TAFII105 | Y09321 | 1.19 | 1.45 | 0.818176516 |
| Tal-1 | NM_003189 | 1.37 | 1.51 | 0.902488517 |
| Tal-1 | NM_003189 | 1.27 | 1.53 | 0.828923165 |
| TARBP2 | NM_004178 | 1.09 | 1.24 | 0.873471591 |
| TARBP2 | NM_004178 | 1.08 | 1.37 | 0.786576406 |
| TBP | NM_003194 | 2.77 | 2.67 | 1.040623399 |
| TBP | NM_003194 | 2.51 | 2.58 | 0.973841382 |
| TBPL1 | NM_004865 | 1.28 | 1.30 | 0.987855849 |
| TBPL1 | NM_004865 | 1.11 | 1.40 | 0.794804113 |
| TBR1 | NM_006593 | 1.19 | 1.20 | 0.996692807 |
| TBR1 | NM_006593 | 1.05 | 1.26 | 0.836636729 |
| TBX19 | NM_005149 | 1.36 | 1.48 | 0.923280344 |
| TBX19 | NM_005149 | 1.44 | 1.59 | 0.909429873 |
| TBX2 | NM_005994 | 0.85 | 1.07 | 0.798962867 |
| TBX2 | NM_005994 | 0.83 | 1.06 | 0.785853316 |
| TBX20 | AJ237589 | 1.34 | 1.21 | 1.102533818 |
| TBX20 | AJ237589 | 1.36 | 1.64 | 0.831910222 |
| TBX6 | NM_004608 | 4.15 | 4.62 | 0.899551242 |
| TBX6 | NM_004608 | 3.96 | 4.53 | 0.875140298 |
| TCEA1 | NM_006756 | 1.27 | 1.14 | 1.110125734 |
| TCEA1 | NM_006756 | 1.16 | 1.11 | 1.05196336 |
| TCEB2 | NM_007108 | 0.58 | 0.43 | 1.337134151 |
| TCEB2 | NM_007108 | 0.51 | 0.40 | 1.277713489 |
| TCF-1 | Z47365 | 1.00 | 1.16 | 0.86404602 |
| TCF-1 | Z47365 | 0.92 | 1.18 | 0.774647829 |
| TCF-4 | Y11306 | 1.36 | 1.46 | 0.928464375 |
| TCF-4 | Y11306 | 1.32 | 1.56 | 0.849608167 |
| TCF21 | NM_003206 | 1.75 | 2.02 | 0.863693344 |
| TCF21 | NM_003206 | 1.69 | 2.12 | 0.798699048 |
| TCF4 | NM_003199 | 0.93 | 0.92 | 1.021001263 |
| TCF4 | NM_003199 | 0.84 | 0.92 | 0.912194059 |
| TCF6L1 | NM_003201 | 1.67 | 1.95 | 0.857799865 |
| TCF6L1 | NM_003201 | 1.82 | 2.34 | 0.776883554 |
| TCFL1 | NM_005997 | 1.45 | 1.63 | 0.891060583 |
| TCFL1 | NM_005997 | 1.45 | 1.68 | 0.863907712 |
| TCFL5 | NM_006602 | 1.87 | 2.31 | 0.809256058 |
| TCFL5 | NM_006602 | 1.79 | 2.27 | 0.788010751 |
| TEAD1 | M63896 | 1.97 | 2.40 | 0.821174945 |
| TEAD1 | M63896 | 1.84 | 2.35 | 0.783305005 |
| TEF-4 | X94440 | 1.14 | 1.29 | 0.883210896 |
| TEF-4 | X94440 | 1.13 | 1.33 | 0.854457478 |
| TF | U79243 | 1.42 | 1.54 | 0.919800195 |
| TF | U79243 | 1.29 | 1.63 | 0.789904601 |
| TFCP2 | NM_005653 | 0.99 | 1.11 | 0.887949768 |
| TFCP2 | NM_005653 | 0.94 | 1.13 | 0.832221275 |
| TFE3 | AL161985 | 1.20 | 1.25 | 0.952888449 |
| TFE3 | AL161985 | 1.16 | 1.30 | 0.896818053 |
| TFIIA | NM_015859 | 0.84 | 0.82 | 1.018380452 |
| TFIIA | NM_015859 | 0.80 | 0.82 | 0.977671128 |
| TFIID | Z22828 | 2.50 | 2.34 | 1.068758898 |
| TFIID | Z22828 | 2.55 | 2.73 | 0.936976254 |
| TFIIH-cyclin H | U11791 | 1.28 | 0.95 | 1.34843684 |
| TFIIH-cyclin H | U11791 | 1.29 | 0.98 | 1.318842969 |
| TFIIH-MO15 | X77743 | 2.43 | 2.39 | 1.014330459 |
| TFIIH-MO15 | X77743 | 2.43 | 2.39 | 1.012865369 |
| TFIIH-p34 | Z30093 | 2.74 | 2.96 | 0.92722107 |
| TFIIH-p34 | Z30093 | 2.34 | 2.95 | 0.792209645 |
| TFRC | NM_003234 | 1.33 | 1.49 | 0.895571075 |
| TFRC | NM_003234 | 1.28 | 1.59 | 0.809200973 |
| TGIF | NM_003244 | 1.75 | 1.38 | 1.274809288 |
| TGIF | NM_003244 | 1.52 | 1.51 | 1.004868421 |
| TIEG2 | NM_003597 | 1.39 | 1.48 | 0.938352308 |
| TIEG2 | NM_003597 | 1.35 | 1.51 | 0.889349637 |
| TIF1GAMMA | NM_015906 | 1.01 | 1.27 | 0.800846532 |
| TIF1GAMMA | NM_015906 | 1.01 | 1.30 | 0.777179202 |
| TIF2 | X97674 | 1.33 | 1.45 | 0.922316636 |
| TIF2 | X97674 | 1.31 | 1.45 | 0.902108121 |
| TIM44 | NM_006351 | 1.33 | 1.57 | 0.847735407 |
| TIM44 | NM_006351 | 1.36 | 1.61 | 0.84323103 |
| Timeless | AF098162 | 2.00 | 2.39 | 0.833636058 |
| Timeless | AF098162 | 1.93 | 2.42 | 0.796169622 |
| TIMM8b | AF152350 | 1.40 | 1.42 | 0.984849477 |
| TIMM8b | AF152350 | 1.42 | 1.51 | 0.941288783 |
| TIMM9 | NM_012460 | 0.71 | 0.77 | 0.920513309 |
| TIMM9 | NM_012460 | 0.68 | 0.84 | 0.805700618 |
| Tis11d | U07802 | 1.12 | 1.35 | 0.827564107 |
| Tis11d | U07802 | 1.04 | 1.27 | 0.821167897 |
| TNRC11 | NM_005120 | 0.88 | 1.05 | 0.836486567 |
| TNRC11 | NM_005120 | 0.85 | 1.05 | 0.802230359 |
| TOB1 | NM_005749 | 1.02 | 1.29 | 0.790953507 |
| TOB1 | NM_005749 | 1.02 | 1.32 | 0.77856472 |
| TOP1 | U07806 | 3.49 | 3.40 | 1.026563028 |
| TOP1 | U07806 | 3.18 | 3.15 | 1.008577791 |
| TP53BP1 | NM_005657 | 0.83 | 0.86 | 0.969466553 |
| TP53BP1 | NM_005657 | 0.79 | 0.86 | 0.910836728 |
| TP73 | NM_005427 | 4.36 | 4.73 | 0.923146915 |
| TP73 | NM_005427 | 3.95 | 4.55 | 0.867754692 |
| TR2 | AF171055 | 1.60 | 1.59 | 1.007806633 |
| TR2 | AF171055 | 1.53 | 1.69 | 0.90288023 |
| TRAF6 | NM_004620 | 1.25 | 1.38 | 0.902520712 |
| TRAF6 | NM_004620 | 1.33 | 1.62 | 0.825230675 |
| TTF-1 | U43203 | 1.57 | 1.92 | 0.818730798 |
| TTF-1 | U43203 | 1.59 | 1.97 | 0.8042506 |
| TTF-I interacting | AF000560 | 0.93 | 1.01 | 0.912809508 |
| peptide | | | | |
| TTF-I interacting | AF000560 | 0.92 | 1.02 | 0.908180051 |
| peptide | | | | |
| TTF1 | NM_007344 | 1.36 | 1.32 | 1.03206288 |
| TTF1 | NM_007344 | 1.21 | 1.33 | 0.908191402 |
| TTP | M63625 | 1.47 | 1.69 | 0.871059069 |
| TTP | M63625 | 1.45 | 1.78 | 0.812551459 |
| tumor suppressor | AJ224819 | 0.97 | 0.96 | 1.010680445 |
| tumor suppressor | AJ224819 | 0.94 | 0.94 | 1.002997158 |
| twist | X91662 | 1.15 | 1.30 | 0.889607419 |
| twist | X91662 | 1.14 | 1.32 | 0.862171118 |
| TZFP | NM_014383 | 1.61 | 1.57 | 1.026900096 |
| TZFP | NM_014383 | 1.31 | 1.62 | 0.806125978 |
| ubiquitin | M26880 | 1.25 | 1.29 | 0.968558812 |
| ubiquitin | M26880 | 1.20 | 1.38 | 0.866123555 |
| UBP1 | NM_014517 | 1.16 | 1.39 | 0.837548498 |
| UBP1 | NM_014517 | 1.05 | 1.31 | 0.801834157 |
| UKLF | AB015132 | 0.94 | 1.15 | 0.812199016 |
| UKLF | AB015132 | 0.91 | 1.13 | 0.807110731 |
| UsF1 | X55666 | 0.92 | 0.77 | 1.202641159 |
| UsF1 | X55666 | 0.90 | 1.16 | 0.779296001 |
| UsF2 | X90824 | 1.70 | 1.51 | 1.12444728 |
| UsF2 | X90824 | 1.49 | 1.47 | 1.010033818 |
| UTF1 | NM_003577 | 0.78 | 0.92 | 0.852557876 |
| UTF1 | NM_003577 | 0.75 | 0.88 | 0.846901451 |
| Vax-2 | Y17791 | 1.95 | 1.73 | 1.125466134 |
| Vax-2 | Y17791 | 1.50 | 1.58 | 0.944890049 |
| VDR | NM_000376 | 2.14 | 1.94 | 1.102535767 |
| VDR | NM_000376 | 2.17 | 1.98 | 1.096166462 |
| Vimentin | X56134 | 0.85 | 0.82 | 1.03779146 |
| Vimentin | X56134 | 0.77 | 0.78 | 0.995470101 |
| VSX1 | NM_014588 | 1.19 | 1.38 | 0.862794625 |
| VSX1 | NM_014588 | 1.14 | 1.36 | 0.838036984 |
| WAVE2 | AB026542 | 1.37 | 1.57 | 0.873152446 |
| WAVE2 | AB026542 | 1.34 | 1.56 | 0.8602453 |
| Whn | Y11746 | 0.95 | 1.05 | 0.89877812 |
| Whn | Y11739 | 0.98 | 1.10 | 0.8889781 |
| winged-helix | AF055080 | 1.80 | 1.62 | 1.112375194 |
| TFforkhead 5 | | | | |
| winged-helix | AF055080 | 1.64 | 1.65 | 0.995925632 |
| TFforkhead 5 | | | | |
| XB | U52701 | 0.93 | 1.00 | 0.931696975 |
| XB | U52701 | 0.86 | 0.99 | 0.874287286 |
| XBP1 | NM_005080 | 1.32 | 1.48 | 0.894006132 |
| XBP1 | NM_005080 | 1.29 | 1.50 | 0.861985033 |
| XG | Z48514 | 0.94 | 1.07 | 0.879021004 |
| XG | Z48514 | 0.94 | 1.11 | 0.844934408 |
| XPE-BF | U32986 | 1.23 | 1.06 | 1.157546744 |
| XPE-BF | U32986 | 1.11 | 1.18 | 0.939655721 |
| XPOT | NM_007235 | 0.93 | 1.04 | 0.888739099 |
| XPOT | NM_007235 | 0.91 | 1.09 | 0.833312043 |
| YAF2 | U72209 | 1.78 | 1.60 | 1.115424048 |
| YAF2 | U72209 | 1.29 | 1.55 | 0.831250433 |
| YPT3 | X79780 | 1.04 | 1.04 | 0.999551657 |
| YPT3 | X79780 | 0.91 | 1.09 | 0.841560488 |
| YWHAZ | NM_003406 | 1.42 | 1.48 | 0.955552146 |
| YWHAZ | NM_003406 | 1.34 | 1.45 | 0.924713154 |
| ZFD25 | AB027251 | 1.38 | 1.47 | 0.935259419 |
| ZFD25 | AB027251 | 1.38 | 1.59 | 0.867549237 |
| ZFM1 | D26120 | 1.38 | 1.52 | 0.904756638 |
| ZFM1 | D26120 | 1.32 | 1.51 | 0.870056053 |
| ZFN3 | X60153 | 1.11 | 1.27 | 0.873020321 |
| ZFN3 | X60153 | 1.08 | 1.27 | 0.84639825 |
| ZFN5128 | NM_014347 | 1.68 | 1.48 | 1.132667677 |
| ZFN5128 | NM_014347 | 1.69 | 1.51 | 1.116327465 |
| ZFP161 | NM_003409 | 1.54 | 1.51 | 1.021814742 |
| ZFP161 | NM_003409 | 1.53 | 1.56 | 0.977142745 |
| ZFP36 | NM_003407 | 1.35 | 1.21 | 1.119420521 |
| ZFP36 | NM_003407 | 1.40 | 1.26 | 1.107958549 |
| ZFP37 | NM_003408 | 2.85 | 3.53 | 0.806477053 |
| ZFP37 | NM_003408 | 3.00 | 3.78 | 0.795656333 |
| ZFS-2 | D70832 | 1.25 | 1.31 | 0.960341853 |
| ZFS-2 | D70832 | 1.19 | 1.34 | 0.887098454 |
| zinc finger factor GKLF | AF105036 | 2.60 | 2.44 | 1.066684361 |
| zinc finger factor GKLF | AF105036 | 2.21 | 2.60 | 0.850960542 |
| ZK1 | NM_005815 | 1.09 | 1.29 | 0.849600519 |
| ZK1 | NM_005815 | 1.13 | 1.34 | 0.848599298 |
| ZMPSTE24 | NM_005857 | 1.59 | 1.96 | 0.807467602 |
| ZMPSTE24 | NM_005857 | 1.64 | 2.04 | 0.804181945 |
| ZNF | AF024700 | 1.37 | 1.42 | 0.968200406 |
| ZNF | AF024700 | 1.25 | 1.43 | 0.877660819 |
| ZNF | AF024702 | 2.44 | 2.27 | 1.071242218 |
| ZNF | AF024702 | 2.13 | 2.46 | 0.864203489 |
| ZNF | AF024708 | 0.85 | 1.06 | 0.803894737 |
| ZNF | AF024708 | 0.88 | 1.10 | 0.795504238 |
| ZNF | AF244088 | 0.96 | 1.18 | 0.80824225 |
| ZNF | AL359576 | 1.96 | 2.40 | 0.816139265 |
| ZNF | AL359576 | 1.97 | 2.45 | 0.804419877 |
| ZNF | L14787 | 0.81 | 0.94 | 0.858334842 |
| ZNF | L14787 | 0.77 | 0.99 | 0.776247563 |
| ZNF | L14843 | 1.04 | 1.21 | 0.859382421 |
| ZNF | L14843 | 1.00 | 1.22 | 0.819499696 |
| ZNF | M77171 | 0.87 | 1.07 | 0.819786317 |
| ZNF | M77171 | 0.84 | 1.06 | 0.788842997 |
| ZNF | M77172 | 1.11 | 1.29 | 0.86138076 |
| ZNF | M77172 | 1.10 | 1.31 | 0.841409825 |
| ZNF | U69645 | 1.02 | 1.11 | 0.923798753 |
| ZNF | U69645 | 1.01 | 1.10 | 0.92309697 |
| ZNF | U90919 | 1.91 | 2.41 | 0.791009945 |
| ZNF | X16282 | 1.04 | 1.06 | 0.976817907 |
| ZNF | X16282 | 0.97 | 1.08 | 0.896468376 |
| ZNF H140 | U80232 | 1.31 | 1.38 | 0.951857602 |
| ZNF H140 | U80232 | 1.25 | 1.46 | 0.852470368 |
| ZNF RIZ | U17838 | 1.37 | 1.49 | 0.919856161 |
| ZNF RIZ | U17838 | 1.33 | 1.52 | 0.878560838 |
| ZNF10 | NM_003419 | 1.14 | 1.24 | 0.918736842 |
| ZNF10 | NM_003419 | 1.06 | 1.25 | 0.852019812 |
| ZNF124 | NM_003431 | 1.66 | 1.70 | 0.976675202 |
| ZNF124 | NM_003431 | 1.69 | 1.79 | 0.942839506 |
| ZNF131 | U09410 | 4.06 | 3.16 | 1.287565844 |
| ZNF131 | U09410 | 3.52 | 3.10 | 1.135482919 |
| ZNF132 | NM_003433 | 1.58 | 1.92 | 0.819592954 |
| ZNF132 | NM_003433 | 1.39 | 1.80 | 0.770618048 |
| ZNF133 | NM_003434 | 1.00 | 1.08 | 0.92665805 |
| ZNF133 | NM_003434 | 0.98 | 1.07 | 0.917239309 |
| ZNF133 | U09366 | 1.59 | 1.48 | 1.074070972 |
| ZNF133 | U09366 | 1.44 | 1.60 | 0.901250209 |
| ZNF134 | NM_003435 | 0.87 | 1.03 | 0.842143011 |
| ZNF134 | NM_003435 | 0.89 | 1.11 | 0.803684073 |
| ZNF135 | NM_003436 | 1.21 | 1.31 | 0.927158596 |
| ZNF136 | NM_003437 | 1.76 | 1.84 | 0.953483104 |
| ZNF136 | NM_003437 | 1.71 | 1.96 | 0.871707485 |
| ZNF139 | U09848 | 1.82 | 2.14 | 0.854333258 |
| ZNF139 | U09848 | 1.88 | 2.25 | 0.83696718 |
| ZNF140 | NM_003440 | 1.18 | 1.31 | 0.902148538 |
| ZNF140 | NM_003440 | 1.10 | 1.38 | 0.795943758 |
| ZNF141 | NM_003441 | 0.94 | 1.10 | 0.852952765 |
| ZNF141 | NM_003441 | 0.97 | 1.14 | 0.851266923 |
| ZNF143 | NM_003442 | 1.23 | 1.38 | 0.888983121 |
| ZNF143 | NM_003442 | 1.25 | 1.41 | 0.88328255 |
| ZNF144 | NM_007144 | 1.39 | 1.50 | 0.928320948 |
| ZNF144 | NM_007144 | 1.37 | 1.47 | 0.926533504 |
| ZNF146 | NM_007145 | 2.27 | 2.48 | 0.916337136 |
| ZNF146 | NM_007145 | 2.13 | 2.64 | 0.805090416 |
| ZNF154 | U20648 | 1.01 | 1.10 | 0.91658232 |
| ZNF154 | U20648 | 0.98 | 1.10 | 0.890033893 |
| ZNF157 | NM_003446 | 3.06 | 3.63 | 0.842644802 |
| ZNF157 | NM_003446 | 3.32 | 4.07 | 0.815095843 |
| ZNF169 | U28251 | 1.07 | 1.06 | 1.007079353 |
| ZNF169 | U28251 | 0.99 | 1.12 | 0.884849008 |
| ZNF173 | NM_003449 | 1.76 | 1.84 | 0.954247529 |
| ZNF173 | NM_003449 | 1.69 | 1.91 | 0.885869838 |
| ZNF174 | U31248 | 1.05 | 1.11 | 0.946809357 |
| ZNF174 | U31248 | 0.98 | 1.07 | 0.916147357 |
| ZNF175 | NM_007147 | 1.42 | 1.58 | 0.896913462 |
| ZNF175 | NM_007147 | 1.37 | 1.66 | 0.824940576 |
| ZNF177 | NM_003451 | 1.35 | 1.31 | 1.030771696 |
| ZNF177 | NM_003451 | 1.16 | 1.36 | 0.850314531 |
| ZNF180 | NM_013256 | 0.97 | 1.13 | 0.856084978 |
| ZNF180 | NM_013256 | 0.97 | 1.18 | 0.824583936 |
| ZNF186 | NM_012480 | 1.05 | 1.14 | 0.913656108 |
| ZNF186 | NM_012480 | 0.99 | 1.11 | 0.892164294 |
| ZNF191 | AF016052 | 3.91 | 4.38 | 0.892949504 |
| ZNF191 | AF016052 | 4.30 | 5.22 | 0.823076704 |
| ZNF200 | NM_003454 | 1.53 | 1.36 | 1.124250225 |
| ZNF200 | NM_003454 | 1.47 | 1.43 | 1.02397083 |
| ZNF211 | NM_006385 | 2.54 | 2.24 | 1.133216101 |
| ZNF211 | NM_006385 | 2.36 | 2.13 | 1.105677798 |
| ZNF214 | NM_013249 | 1.12 | 1.35 | 0.833321399 |
| ZNF214 | NM_013249 | 1.15 | 1.43 | 0.806432749 |
| ZNF215 | NM_013250 | 1.09 | 1.24 | 0.879083204 |
| ZNF215 | NM_013250 | 1.13 | 1.35 | 0.837769383 |
| ZNF216 | AF062073 | 6.28 | 6.85 | 0.916789497 |
| ZNF216 | AF062073 | 6.12 | 6.94 | 0.882430365 |
| ZNF22 | NM_006963 | 1.06 | 1.27 | 0.835382221 |
| ZNF22 | NM_006963 | 1.05 | 1.29 | 0.80987367 |
| ZNF220 | NM_006766 | 1.16 | 1.31 | 0.88697634 |
| ZNF220 | NM_006766 | 1.15 | 1.35 | 0.848209348 |
| ZNF223 | NM_013361 | 1.29 | 1.44 | 0.90055118 |
| ZNF223 | NM_013361 | 1.25 | 1.45 | 0.865757643 |
| ZNF228 | NM_013380 | 1.13 | 1.10 | 1.028423144 |
| ZNF228 | NM_013380 | 0.84 | 1.00 | 0.838339028 |
| ZNF229 | AF192979 | 1.44 | 1.74 | 0.826578529 |
| ZNF229 | AF192979 | 1.41 | 1.76 | 0.802898585 |
| ZNF231 | NM_003458 | 1.29 | 1.38 | 0.933778707 |
| ZNF231 | NM_003458 | 1.24 | 1.42 | 0.875912367 |
| ZNF232 | NM_014519 | 3.91 | 3.54 | 1.103967871 |
| ZNF232 | NM_014519 | 3.65 | 3.40 | 1.074957509 |
| ZNF232 | AF080171 | 0.94 | 1.07 | 0.871430609 |
| ZNF232 | AF080171 | 0.92 | 1.08 | 0.856315576 |
| ZNF258 | NM_007167 | 3.65 | 2.56 | 1.429969861 |
| ZNF258 | NM_007167 | 2.86 | 2.23 | 1.280364117 |
| ZNF261 | NM_005096 | 2.03 | 2.19 | 0.929608653 |
| ZNF261 | NM_005096 | 1.79 | 2.05 | 0.873798627 |
| ZNF297 | NM_005453 | 1.40 | 1.66 | 0.844649054 |
| ZNF297 | NM_005453 | 1.39 | 1.65 | 0.841507575 |
| ZNF31 | U71600 | 0.91 | 1.13 | 0.804205241 |
| ZNF31 | U71600 | 0.89 | 1.16 | 0.770222697 |
| ZNF35 | NM_003420 | 1.53 | 1.57 | 0.977397734 |
| ZNF35 | NM_003420 | 1.52 | 1.62 | 0.938058006 |
| ZNF37A | X69115 | 0.96 | 1.05 | 0.915518905 |
| ZNF37A | X69115 | 0.94 | 1.13 | 0.835188396 |
| ZNF41 | M92443 | 1.52 | 1.91 | 0.798258529 |
| ZNF41 | M92443 | 1.53 | 1.99 | 0.771414141 |
| ZNF41 | X60155 | 0.99 | 1.06 | 0.934017616 |
| ZNF41 | X60155 | 1.01 | 1.10 | 0.917673489 |
| ZNF47 | U71601 | 1.03 | 1.18 | 0.872466879 |
| ZNF47 | U71601 | 0.96 | 1.14 | 0.8440485 |
| ZNF7 | NM_003416 | 1.04 | 1.17 | 0.895223602 |
| ZNF7 | NM_003416 | 1.07 | 1.23 | 0.869409968 |
| ZNF8 | M29581 | 0.94 | 1.00 | 0.940042921 |
| ZNF8 | M29581 | 0.84 | 0.94 | 0.89819251 |
| ZNF80 | NM_007136 | 2.05 | 1.92 | 1.064684732 |
| ZNF80 | NM_007136 | 1.98 | 1.93 | 1.024108326 |
| ZNF85 | NM_003429 | 1.27 | 1.39 | 0.915903902 |
| ZNF85 | NM_003429 | 1.14 | 1.42 | 0.797896136 |
| ZNF91 | NM_003430 | 1.09 | 1.09 | 1.002040082 |
| ZNF91 | NM_003430 | 1.04 | 1.07 | 0.975112741 |
| ZNFB7 | U34249 | 1.41 | 1.47 | 0.955940013 |
| ZNFB7 | U34249 | 1.34 | 1.50 | 0.893216374 |
| ZNFN1A3 | NM_012481 | 1.17 | 1.06 | 1.108208901 |
| ZNFN1A3 | NM_012481 | 1.17 | 1.06 | 1.100020165 |
| ZNK75a | X91826 | 0.99 | 1.18 | 0.840732485 |
| ZNK75a | X91826 | 0.98 | 1.24 | 0.792918773 |
| ZRP-1 | AF000974 | 4.16 | 4.74 | 0.87742034 |
| ZRP-1 | AF000974 | 4.06 | 4.99 | 0.813590373 |
| ZYX | NM_003461 | 1.40 | 1.36 | 1.031031823 |
| ZYX | NM_003461 | 1.31 | 1.36 | 0.963983275 |

List of References

1. Chen, Y., Dougherty, E. R., Bittner, M. L. (1997) J. Biomed. Optics 24: 364-374.

2. Hegde, P., Qi, R., Abernathy, K., Gay, C., Dharap, S., Gaspard, R., Earle-Hughes, J., Snesrud, E., Lee, N., Quackenbush, J. (2000) Biotechniques 29:548-562.

3. Yang M C, Ruan Q G, Yang J J, Eckenrode S, Wu S, McIndoe R A, She J X. (2001) Physiol Genomics 7:45-53.

4. Schena M, Shalon D, Davis R W, Brown P O. (1995). Science 270:467-70.

5. Cho Y J, Meade J D, Walden J C, Chen X, Guo Z, Liang P. (2001) Biotechniques 30:562-8, 570, 572

6. Brown A J, Planta R J, Restuhadi F, Bailey D A, Butler P R, Cadahia J L, Cerdan M E, De Jonge M, Gardner D C, Gent M E, Hayes A, Kolen C P, Lombardia L J, Murad A M, Oliver R A, Sefton M, Thevelein J M, Tournu H, van Delft Y J, Verbart D J, Winderickx J, Oliver S G. (2001). EMBO J 20:3177-86.

7. Chaib H, Cockrell E K, Rubin M A, Macoska J A. (2001) Neoplasia:43-52.

8. Barbu V, Dautry F. (1989) Nucleic Acids Res17:7115

9. Gaudette M F, Crain W R. (1991). Nucleic Acids Res. 19:1879-84.

10. Horikoshi S, Fukuda K, Ray P E, Sawada M, Bruggeman L A, Klotman P E. (1992). Kidney Int. 42:764-9.

11. Kerr M K, Churchill G A. (2001). Proc Natl Acad Sci U S A 98:8961-5

What is claimed is:

1. A method for providing an internal standard for normalizing the relative intensities of signals on a hybridization array, comprising:
adding a known quantity of an unlabelled ribosomal nucleic acid competitor probe into a hybridization buffer suitable for the array experiment, the competitor probe characterized in that it has the same as a portion of a capture probe present in the array for immobilizing ribosomal nucleic acids thereon; and
allowing the competitor probe to compete with a ribosomal capture probe for hybridization to a suitably labelled rRNA-derived cDNA of a cDNA sample, such that a hybridization signal of labelled rRNA-derived cDNA is decreased to a suitable signal dynamic range of detection and the rRNA-derived cDNA of the sample becomes a suitable internal standard for the hybridization array.

2. A method for normalizing the relative intensities of signals on a hybridization array, comprising:
reproducing the method of claim 1 with a first reference sample labelled with a first label, and with a second test sample labelled with a second label; and
comparing the intensity of a hybridization signal of hybridized rRNA-derived cDNA originating from the test sample to the intensity of a hybridization signal of hybridized rRNA-derived cDNA originating from the reference sample, to obtain a normalization factor.

3. A hybridization assay comprising:
reproducing the method of claim 2; and
normalizing the signals provided for each label for a given target nucleic acid hybridizing to a target-specific capture probe, said target originating from the reference and being labelled with the first label and from the test sample and being labelled with the second label, with the normalization factor.

4. A method as defined in any one of claims 1 to 3, further comprising:
determining the quantity of hybridized rRNA-derived cDNA.

5. A method as defined in claim 4, further comprising:
comparing the quantity of hybridized rRNA-derived cDNA against standard curves to determine the quantity of cDNA in said sample.

6. A method as described in any one of claims 1 to 5, wherein said rRNA competitor probe is present in a concentration that is about 5 to about 100 times that of the rRNA-cDNA probe.

7. A method as described in anyone of claims 1 to 6, wherein said rRNA-derived cDNA is labelled by 3′ addition of phosphate, cyanines, biotin, digoxygenin, fluorescein, a dideoxynucleotide, an amine, a thiol, an azo (N₃) group, fluorine, or any other form of label.

8. A method as described in any one of claims 1 to 7, which is used in high-throughput screening.

9. A method as described in any one of claims 1 to 8, wherein said array experiment consists in the identification of sequences found in the open reading frame of genes coding for transcription factors.

10. A method as described in claim 8, wherein said transcription factors include c-Rel, E2F-1, Egr-1, ER, NFκB p50, p53, Sp1 and YY1.

11. A solid support displaying an array of probes bound thereto, which array comprises a capture probe complementary to ribosomal nucleic acids or to cDNA derived therefrom.

12. A hybridization kit which comprises the solid support of claim 11 and, as a separate component, a competitor probe, the sequence of which comprises a least a portion of the sequence of the capture probe.


SEQUENCE LISTING (3 sequences)

SEQ ID NO: 1 - DNA, 21 bp, Artificial Sequence
  Description of Artificial Sequence Oligonucleotide
cttatgacccgcacttactcg

SEQ ID NO: 2 - DNA, 40 bp, Artificial Sequence
  Description of Artificial Sequence Oligonucleotide
cccgagccgcctggataccgcagctaggaataatggaata

SEQ ID NO: 3 - DNA, 40 bp, Artificial Sequence
  Description of Artificial Sequence Oligonucleotide
tctcgattccgtgggtggtggtgcatggccgttcttagtt